(19) United States
(12) Patent Application Publication
(10) Pub. No.: US 2025/0292405 A1
(43) Pub. Date: Sep. 18, 2025

(54) Gum Line Extraction Method, Manufacturing Method of Dental Device, and Electronic Device

(71) Applicant: GUANGZHOU HEYGEARS IMC. INC, Guangzhou (CN)

(72) Inventors: Jinhai ZHOU, Guangzhou (CN); Dachao ZHOU, Guangzhou (CN); Yong WANG, Guangzhou (CN); Miao DENG, Guangzhou (CN)

(73) Assignee: GUANGZHOU HEYGEARS IMC. INC, Guangzhou (CN)

(21) Appl. No.: 19/225,754

(22) Filed: Jun. 2, 2025

Related U.S. Application Data
(63) Continuation-in-part of application No. PCT/CN2023/131076, filed on Nov. 10, 2023.

(30) Foreign Application Priority Data
Dec. 2, 2022 (CN) 202211543176.2
May 4, 2023 (CN) 202310493978.5

(51) Int. Cl. G06T 7/00; G06T 7/13

(52) U.S. Cl. CPC G06T 7/0012; G06T 7/13; G06T 2200/04; G06T 2207/30036

(57) ABSTRACT

A gum line extraction method, a manufacturing method of a dental device, and an electronic device are provided. The gum line extraction method includes: obtaining a three-dimensional tooth model, the three-dimensional teeth model including a plurality of polygonal patches; determining an edge category of an edge of the plurality of polygonal patches, the edge category including a tooth edge and a gum edge; and determining a gum line of the three-dimensional tooth model according to the edge category of the edge of the plurality of polygonal patches.

### TECHNICAL FIELD

The present disclosure relates to the technical field of dental orthodontics, and in particular to a gum line extraction method, a manufacturing method of a dental device, and an electronic device.

### BACKGROUND

In the current application scenarios of invisible orthodontic tooth diagnosis and treatment, all application scenarios require data processing of tooth models, and data pre-processing of the tooth models occupies most of the time of a 3D tooth printing work. Especially in the application scenario of orthodontic correction, in addition to performing data pre-processing on the tooth model, including placement, hollowing out and a Boolean operation, operations such as film pressing and cutting are further required after printing is completed, wherein the cutting operation is performed based on a gum line.

Currently, the gum line is mainly acquired by manual drawing or automatic identification, or the like, however, the applicant finds that the accuracy and efficiency of gum lines acquired based on the existing gum line acquisition methods are relatively low, and thus being difficult to meet the use requirements.

### SUMMARY

The present disclosure provides a gum line extraction method, a manufacturing method of a dental device, and an electronic device, so as to solve the technical problem in the prior art that a gum line of a tooth model cannot be accurately and efficiently extracted.

The present disclosure provides a gum line extraction method, including: obtaining a three-dimensional tooth model, wherein the three-dimensional tooth model includes a plurality of polygonal patches; determining an edge category of an edge of the plurality of polygonal patches, wherein the edge category includes a tooth edge and a gum edge; and determining a gum line of the three-dimensional tooth model according to the edge category of the edge of the plurality of polygonal patches.

The present disclosure provides a manufacturing method of a dental device, including: after a gum line is acquired according to the above gum line extraction method, converting the gum line into a cutting line; and controlling cutting of an initial equipment based on the cutting line to obtain the dental device, wherein the initial equipment has an association relationship with a three-dimensional tooth model.

As an optional example, the manufacturing method of the dental device further includes: performing tooth cutting on the three-dimensional tooth model.

As an optional example, the manufacturing method of the dental device further includes: performing arrangement processing on teeth to obtain a tooth model to be formed, wherein the initial equipment has an association relationship with the tooth model to be formed.

As an optional example, the manufacturing method of the dental device further includes: performing tooth cutting on the three-dimensional tooth model; and performing arrangement processing on teeth obtained after tooth cutting, to obtain the tooth model to be formed, wherein the initial equipment has an association relationship with the tooth model to be formed.

The present disclosure provides a three-dimensional tooth model segmentation method, including: obtaining a two-dimensional projection image of a three-dimensional tooth model, and identifying the two-dimensional projection image to obtain a plurality of tooth regions; determining, in the three-dimensional tooth model, original seed points corresponding to the plurality of tooth regions; expanding the original seed points within a preset range to obtain target seed points of teeth in the three-dimensional tooth model; and segmenting the three-dimensional tooth model based on the target seed points of the teeth to obtain cut teeth.

As an optional example, the expanding the original seed points within the preset range to obtain the target seed points of the teeth in the three-dimensional tooth model includes: expanding the original seed points according to preset curvature thresholds to obtain the target seed points.

As an optional example, the expanding the original seed points within the preset range to obtain the target seed points of the teeth in the three-dimensional tooth model includes: expanding the original seed points according to initial curvature thresholds to obtain first seed points; and expanding the first seed points according to a target curvature threshold to obtain the target seed points, wherein the target curvature threshold is obtained according to the initial curvature threshold.

As an optional example, expanding the original seed points according to the preset curvature thresholds to obtain the target seed points includes: expanding the original seed points according to the initial curvature thresholds to obtain the first seed points; and expanding the first seed points according to a target curvature threshold to obtain the target seed points, wherein the target curvature threshold is obtained according to the initial curvature threshold.

As an optional example, the expanding the original seed points according to the initial curvature thresholds to obtain the first seed points includes:

using seed points adjacent to the original seed points as current seed points; and in a case where the curvatures of the current seed points are less than or equal to the initial curvature threshold, using the current seed points and the original seed points as the first seed points.

As an optional example, the expanding the first seed points according to the target curvature threshold to obtain the target seed points includes: using seed points adjacent to the first seed points as current seed points; and in a case where the curvatures of the current seed points are less than or equal to the target curvature threshold, using the first seed points and the current seed points as the target seed points.

As an optional example, before expanding the first seed points according to the target curvature threshold to obtain the target seed points, the method further includes: using a sum of the initial curvature threshold and a preset value as the target curvature threshold, wherein the preset value is a positive number.

As an optional example, after expanding the original seed points according to the initial curvature threshold to obtain the first seed points, or expanding the first seed points according to the target curvature threshold to obtain the target seed points, the method further includes: adjusting the first seed points as non-first seed points in a case where corresponding points of the first seed points on the two-dimensional projection image do not fall into corresponding tooth regions; or, adjusting the target seed points as non-target seed points in a case where corresponding points of the target seed points on the two-dimensional projection image do not fall into the corresponding tooth regions.

As an optional example, after expanding the original seed points according to the curvature threshold to obtain the target seed points, and before segmenting the three-dimensional tooth model based on the target seed points of the teeth to obtain the cut teeth, the method further includes: expanding the plurality of tooth regions to obtain target regions; and expanding the target seed points in the target regions according to the initial curvature thresholds and heights.

As an optional example, the three-dimensional tooth model segmentation method may be used for performing tooth segmentation processing on the three-dimensional tooth model.

As an optional example, acquiring a fitting feature point of each tooth in the three-dimensional tooth model; performing fitting according to a position relationship of the tooth relative to the three-dimensional tooth model and the fitting feature point, to obtain a dental arch curve and a torque angle curve; determining a target point closest to each fitting feature point on the dental arch curve, and determining a position where the target point is located as a target position of the corresponding tooth; obtaining a target pose of each tooth corresponding to the target position according to the dental arch curve and the torque angle curve; and moving the tooth to the corresponding target position, and adjusting a pose of the tooth to the corresponding target pose.

As an optional example, an acquisition module is configured to acquire a fitting feature point of each tooth in the three-dimensional tooth model; a fitting module is configured to perform fitting according to a position relationship of the tooth relative to the three-dimensional tooth model and the fitting feature point, to obtain a dental arch curve and a torque angle curve; a first determination module is configured to determine a target point closest to each fitting feature point on the dental arch curve, and determine a position where the target point is located as a target position of the corresponding tooth; a second determination module is configured to obtain a target pose of each tooth corresponding to the target position according to the dental arch curve and the torque angle curve; and a moving module is configured to move the tooth to the corresponding target position, and adjust a pose of the tooth to the corresponding target pose.

As an optional example, an apparatus further includes: an establishment module, configured to establish a total coordinate system of the three-dimensional tooth model and a local coordinate system of the tooth; and a third determination module, configured to obtain the position relationship according to the total coordinate system and the local coordinate system.

As an optional example, the apparatus further includes: a fourth determination module, configured to determine that pose adjustment of the tooth is completed in a case where a current state of the tooth meets a preset condition, wherein the preset condition includes: a direction of the tooth on a first coordinate axis of the local coordinate system is consistent with a tangential direction of the dental arch curve at the target point, a direction of the tooth on a second coordinate axis of the local coordinate system is perpendicular to the first coordinate axis, and an angle between the tooth and a first coordinate axis of the total coordinate system meets the torque angle curve.

As an optional example, the fitting module includes: a first fitting unit, configured to determine an x value of the fitting feature point of the tooth, and an included angle between the second coordinate axis of the local coordinate system of the tooth and the first coordinate axis of the total coordinate system; and perform fitting processing on the x value of the fitting feature point and the included angle to obtain the torque angle curve.

As an optional example, the fitting module includes: a second fitting unit, configured to fit and solve a pre-established ideal dental arch curve model according to the fitting feature point, to obtain an initial dental arch curve and a correction parameter, wherein the correction parameter includes a lateral translation amount, a longitudinal translation amount and a rotation angle; and adjust a position of the initial dental arch curve according to the correction parameter, to obtain the dental arch curve, wherein the dental arch curve is located on the ideal dental arch curve model.

As an optional example, the apparatus further includes: a simulation module, configured to: after acquiring the fitting feature point of each tooth in the three-dimensional tooth model, in a case where there is no target tooth on a tooth position of the three-dimensional tooth model, obtain the fitting feature point of the target tooth according to simulation data of the target tooth, wherein the simulation data is obtained by simulating the target tooth by using data of a tooth on a symmetrical tooth position on the same jaw as the target tooth.

As an optional example, the simulation data includes a coordinate of the target tooth; and the apparatus further includes: a fifth determination module, configured to determine a first coordinate value of the tooth on the symmetrical tooth position on the same jaw as the target tooth under the total coordinate system, and determine a second coordinate value as the coordinate of the target tooth, wherein the second coordinate value is symmetrical to the first coordinate value with respect to a target plane, and the target plane is a plane in which the coordinate value of the first coordinate axis of the total coordinate system is zero.

As an optional example, the apparatus further includes: an adjustment module, configured to: before acquiring the fitting feature point of each tooth in the three-dimensional tooth model, adjust the position of the three-dimensional tooth model to a target position, and adjust the orientation of the three-dimensional tooth model to a target orientation.

As an optional example, the adjustment module includes: an adjustment unit, configured to determine a center point, a first vector and a second vector of the three-dimensional tooth model, wherein the first vector and the second vector are configured to adjust the orientation of the three-dimensional tooth model, and the center point is configured to adjust the position of the three-dimensional tooth model; and adjust the three-dimensional tooth model, so that the first vector coincides with a Z axis of the total coordinate system of the three-dimensional tooth model, the second vector coincides with an X axis of the total coordinate system, and that the center point coincides with an origin of the total coordinate system.

As an optional example, the adjustment unit includes: a first determination sub-unit, configured to traverse the teeth of the three-dimensional tooth model in a sequence from an incisor to a last molar of the three-dimensional tooth model, and determine a centroid of each tooth on the three-dimensional tooth model; and use, as the center point, a center of a first centroid connecting line of the centroid of a traversed first tooth having no missing teeth on left and right sides and the centroid of a traversed last tooth having no missing teeth on the left and right sides.

As an optional example, the adjustment unit includes: a second determination sub-unit, configured to traverse the teeth of the three-dimensional tooth model in a sequence from a last molar to the incisor of the three-dimensional tooth model, and determine a centroid of each tooth on the three-dimensional tooth model; determine, as the second vector, a second centroid connecting line of the centroid of a traversed first tooth having no missing teeth on the left and right sides and the centroid of a traversed last tooth having no missing teeth on the left and right sides; determine a center of the second centroid connecting line as an auxiliary point; determine a vector from the auxiliary point to the center point as an auxiliary vector; and determine a product of the second vector and the auxiliary vector as the first vector.

As an optional example, the acquisition module includes: a processing unit, configured to use each tooth as a current tooth; determine a first feature point and a second feature point of the current tooth according to the three-dimensional tooth model; according to a type to which the current tooth belongs, determine a third feature point for the current tooth by using an apparatus matching the type; and determine the fitting feature point of the current tooth from the first feature point, the second feature point and the third feature point.

As an optional example, the processing unit includes: a third determination sub-unit, configured to intercept a target vertex and a polygonal patch from the three-dimensional tooth model after the three-dimensional tooth model is straightened; determine a linear vector of the target vertex; determine a target bounding box of the target vertex and the polygonal patch, wherein an X-axis direction of the target bounding box is a direction of the linear vector; divide the three-dimensional tooth model into a cheek-side part and a tongue-side part according to the target bounding box; calculate a first bounding box of the three-dimensional tooth model of the cheek-side part; and use, as the first feature point and the second feature point of the current tooth, two points closest to two endpoints on a top of the first bounding box in vertexes of the three-dimensional tooth model of the cheek-side part.

As an optional example, the third determination sub-unit is further configured to: in a case where the current tooth is a first-type tooth, use, as the third feature point of the current tooth, a point closest to a bottom of the first bounding box in the vertexes of the current tooth in the three-dimensional tooth model of the cheek-side part; in a case where the current tooth is a second-type tooth, use, as the third feature point of the current tooth, a point closest to a top of the first bounding box in the vertexes of the current tooth in the three-dimensional tooth model of the cheek-side part; in a case where the current tooth is a third-type tooth or a fourth-type tooth, extract a second bounding box of the current tooth; determine a first vertex set and a second vertex set from the second bounding box; determine a pit and fissure direction of the current tooth according to the second vertex set; adjust a direction of the second bounding box to be consistent with the pit and fissure direction; divide the current tooth into four parts according to the adjusted second bounding box; in a case where each part of the current tooth includes the vertexes in the first vertex set, use, as the third feature point of the current tooth, a vertex closest to a top of the second bounding box in the vertexes in the first vertex set; and in a case where each part of the current tooth does not include the vertexes in the first vertex set, traverse the vertexes from one vertex in the current part of the current tooth to a center of the second bounding box, and use a point in the traversed vertexes that is closest to a top of the second bounding box as the third feature point of the current tooth.

As an optional example, the processing unit includes: a fourth determination sub-unit, configured to: in a case where the current tooth is the first-type tooth or the fourth-type tooth, use a midpoint of a connecting line between the first feature point and the second feature point of the current tooth as the fitting feature point; and in a case where the current tooth is the second-type tooth, use the third feature point of the current tooth as the fitting feature point; and in a case where the current tooth is the third-type tooth, use the first feature point of the current tooth as the fitting feature point.

As an optional example, the apparatus further includes: a readjustment module, configured to: after performing fitting according to the position relationship of the tooth relative to the three-dimensional tooth model and the fitting feature point, adjust y coordinate values of the fitting feature points corresponding to the second-type tooth and the third-type tooth according to a preset proportionality coefficient; and perform fitting on the dental arch curve again.

As an optional example, the apparatus further includes: a collision detection module, configured to perform adjustment on the tooth in the X-axis direction via collision detection; adjust the tooth in the Z-axis direction after the adjustment in the X-axis direction; adjust an upper jaw in the Y-axis direction according to an upper jaw and lower jaw occlusion relationship; and adjust the tooth in the Z-axis direction according to the collision detection.

As an optional example, the collision detection module includes: a first collision detection unit, configured to horizontally move a first tooth in each tooth region to a position colliding with the target plane while keeping a pose unchanged, wherein the target plane is a plane in which X=0; use each tooth behind the first tooth in each tooth region as the current tooth, and perform the following operation: in a case where a previous tooth of the current tooth exists, adjust a pose of the current tooth via collision detection between the current tooth and the previous tooth.

As an optional example, the collision detection module includes: a second collision detection unit, configured to determine a Spee curve according to a coordinate of the fitting feature point of each tooth, wherein in a case where there is a missing tooth, simulate a fitting feature point of a tooth-missing position by using a fitting feature point of a tooth on a symmetrical position of the tooth-missing position; according to a Z coordinate value of each tooth on the Spee curve, determine whether to adjust the Spee curve; and adjust the Z-axis direction of the tooth according to the adjusted Spee curve.

As an optional example, the collision detection module includes: a third collision detection unit, configured to move an upper jaw of the three-dimensional tooth model upwards along the Z axis for a predetermined distance, wherein the predetermined distance is a difference value between a fitting feature point with a minimum Z value in the upper jaw and a fitting feature point with a maximum Z value in the lower jaw; and move each tooth in the upper jaw downwards via collision detection until colliding with the teeth in the lower jaw.

As an optional example, the tooth arrangement method of the three-dimensional tooth model may be used for performing arrangement processing on teeth to obtain the tooth model to be formed.

According to another aspect of the embodiments of the present disclosure, provided is an electronic device, including a memory, a processor, a communication interface, and a communications bus, wherein a computer program capable of running on the processor is stored in the memory, the memory and the processor perform communication with the communication interface via the communications bus, and the processor, when executing the computer program, implements the steps of the above methods.

According to another aspect of the embodiments of the present disclosure, further provided is a computer-readable storage medium, wherein the computer-readable storage medium stores a computer program, and the computer program, when operated by a processor, executes the gum line extraction method or the manufacturing method of the shell-shaped dental device.

Compared with related arts, the above technical solutions provided in the embodiments of the present disclosure have the following advantages:

In the present disclosure, after the three-dimensional tooth model is obtained, the gum line of the three-dimensional tooth model may be determined according to the edge categories of the edges by a method for determining the edge categories of the edges of the polygonal patches on the three-dimensional tooth model. The edges, serving as components of the polygonal patches, may further refine the three-dimensional tooth model, so that the accuracy of extracting the gum line of the tooth model can be improved.

In addition, after the gum line is obtained according to the gum line extraction method, the gum line is converted into the cutting line; and the cutting of the initial equipment is controlled based on the cutting line to obtain the dental device, such as an invisible appliance.

Moreover, in the present disclosure, after the three-dimensional tooth model is obtained, the original seed points on the three-dimensional tooth model are determined by identifying the two-dimensional projection image of the three-dimensional tooth model, and the original seed points are expanded to obtain the target seed points, so that the range of the teeth on the three-dimensional tooth model is marked by the target seed points, and the three-dimensional tooth model may be further cut according to the target seed points to obtain the cut teeth, thereby achieving the effect of accurately performing tooth segmentation on the three-dimensional tooth model, and solving the problem in the prior art that the accuracy of performing tooth segmentation on the three-dimensional tooth model is low; and compared with traditional tooth segmentation means, the present disclosure may also avoid segmenting the tooth-missing position.

In addition, in the present disclosure, a method is further used, including: obtaining the fitting feature point of each tooth in the three-dimensional tooth model; performing fitting according to the position relationship of the tooth relative to the three-dimensional tooth model and the fitting feature point, to obtain the dental arch curve and the torque angle curve; determining the target point closest to each fitting feature point on the dental arch curve, and determining the position where the target point is located as the target position of the corresponding tooth; obtaining the target pose of each tooth corresponding to the target position according to the dental arch curve and the torque angle curve; and moving the tooth to the corresponding target position, and adjusting the pose of the tooth to the corresponding target pose. In the method, during the process of performing tooth arrangement on the teeth in the three-dimensional tooth model, the dental arch curve and the torque angle curve may be determined by the fitting feature point, the target point closest to the fitting feature point is determined on the dental arch curve, the tooth is moved to the target point on the dental arch curve, and the pose of the tooth is adjusted to the target pose, thereby achieving the purpose of automatic tooth arrangement, thus solving the technical problem of low manual tooth arrangement efficiency.

### BRIEF DESCRIPTION OF THE DRAWINGS

The drawings herein are incorporated in and constitute a part of the present specification, illustrate embodiments conforming to the present disclosure, and serve to explain the principles of the present disclosure together with the specification.

To illustrate technical solutions in the embodiments of the present disclosure or in the related art more clearly, a brief introduction on the drawings which are needed in the description of the embodiments or the related art is given below. Apparently, for those ordinary skilled in the art, other drawings may also be obtained according to these drawings without creative efforts.

FIG. 1 is a schematic diagram of a hardware environment of an optional gum line extraction method provided according to an embodiment of the present disclosure;

FIG. 2 is a flowchart of an optional gum line extraction method provided according to an embodiment of the present disclosure;

FIG. 3 is a schematic diagram of a polygonal patch of an optional gum line extraction method provided according to an embodiment of the present disclosure;

FIG. 4 is a diagram of an identification model of an optional gum line extraction method provided according to an embodiment of the present disclosure;

FIG. 5 is a schematic diagram of convolution and pooling operations performed based on edges in a three-dimensional tooth model in an optional gum line extraction method provided according to an embodiment of the present disclosure;

FIG. 6 is a flowchart of an optional manufacturing method of a dental device provided according to an embodiment of the present disclosure;

FIG. 7 is a schematic diagram of down sampling of an optional gum line extraction method provided according to an embodiment of the present disclosure;

FIG. 8 is a flowchart of an optional three-dimensional tooth model segmentation method provided according to an embodiment of the present disclosure;

FIG. 9 is a schematic diagram of peak points and valley points of an optional three-dimensional tooth model provided according to an embodiment of the present disclosure;

FIG. 10 is a schematic diagram of tooth segmentation of an optional three-dimensional tooth model provided according to an embodiment of the present disclosure;

FIG. 11 is a flowchart of an optional three-dimensional tooth model provided according to an embodiment of the present disclosure;

FIG. 12 is a schematic diagram of a peak point combination of an optional three-dimensional tooth model provided according to an embodiment of the present disclosure;

FIG. 13 is a segmentation curve graph of an optional three-dimensional tooth model provided according to an embodiment of the present disclosure;

FIG. 14 is a flowchart of an optional three-dimensional tooth model segmentation method provided according to an embodiment of the present disclosure;

FIG. 15 is a two-dimensional projection image of an optional three-dimensional tooth model segmentation method provided according to an embodiment of the present disclosure;

FIG. 16 is a diagram of a tooth region of an optional three-dimensional tooth model segmentation method provided according to an embodiment of the present disclosure;

FIG. 17 is a diagram of a tooth region of another optional three-dimensional tooth model segmentation method provided according to an embodiment of the present disclosure;

FIG. 18 is a diagram of a triangular patch of an optional three-dimensional tooth model segmentation method provided according to an embodiment of the present disclosure;

FIG. 19 is a diagram of a smooth boundary of an optional three-dimensional tooth model segmentation method provided according to an embodiment of the present disclosure;

FIG. 20 is a tooth arrangement diagram of an optional three-dimensional tooth model segmentation method provided according to an embodiment of the present disclosure;

FIG. 21 is a flowchart of an optional tooth arrangement method of a three-dimensional tooth model provided according to an embodiment of the present disclosure;

FIG. 22 is a diagram of a straightening parameter of an optional tooth arrangement method of a three-dimensional tooth model provided according to an embodiment of the present disclosure;

FIG. 23 is a diagram of bottom tip points on a cheek side and a tongue side of an optional tooth arrangement method of a three-dimensional tooth model provided according to an embodiment of the present disclosure;

FIG. 24 is a diagram of a tooth center connecting line vector of an optional tooth arrangement method of a three-dimensional tooth model provided according to an embodiment of the present disclosure;

FIG. 25 is a diagram of three feature points of an incisor of an optional tooth arrangement method of a three-dimensional tooth model provided according to an embodiment of the present disclosure;

FIG. 26 is a diagram of three feature points of a cuspid tooth of an optional tooth arrangement method of a three-dimensional tooth model provided according to an embodiment of the present disclosure;

FIG. 27 is a diagram of a third feature point of a premolar of an optional tooth arrangement method of a three-dimensional tooth model provided according to an embodiment of the present disclosure;

FIG. 28 is a diagram of a third feature point of a posterior molar of an optional tooth arrangement method of a three-dimensional tooth model provided according to an embodiment of the present disclosure;

FIG. 29 is a schematic diagram of a missing tooth of an optional tooth arrangement method of a three-dimensional tooth model provided according to an embodiment of the present disclosure;

FIG. 30 is a schematic structural diagram of an optional tooth arrangement apparatus of a three-dimensional tooth model provided according to an embodiment of the present disclosure; and

FIG. 31 is a schematic diagram of an optional electronic device provided according to an embodiment of the present disclosure.

### DETAILED DESCRIPTION OF THE EMBODIMENTS

In order to make the objectives, technical solutions and advantages of the embodiments of the present disclosure clearer, a clear and complete description of technical solutions in the embodiments of the present disclosure will be given below, in combination with the drawings in the embodiments of the present disclosure. Apparently, the embodiments described below are merely a part, but not all, of the embodiments of the present disclosure. All of other embodiments, obtained by those ordinary skilled in the art based on the embodiments in the present disclosure without any creative effort, fall into the protection scope of the present disclosure.

In the following description, suffixes, such as “modules”, “components” or “units”, used for representing elements are merely for the purpose of facilitating the present disclosure, and have no particular meaning in themselves. Thus, “modules” and “components” may be used in admixture.

3D: Three Dimensions; three-dimensional, abbreviated as 3D

STL: a file format (an abbreviation of stereo lithography), which is an interface protocol formulated by the 3D SYSTEMS company in 1988 and is a three-dimensional graphic file format serving the rapid prototyping technology. An STL file is composed of definitions of a plurality of triangular patches, and the definition of each triangular patch includes a three-dimensional coordinate of each fixed point of a triangle and a normal vector of the triangular patch.

FDI: a dental notation, also referred to as a numerical notation method, is universal in the world. Each tooth is recorded with two Arabic numerals, the first numeral represents a quadrant where the tooth is located: 1, 2, 3 and 4 on the upper right, the upper left, the lower left and the lower right of a patient represent permanent teeth, and 5, 6, 7 and 8 represent deciduous teeth;

and the second numeral represents the position of the tooth: 1-8 represent a central incisor to a third molar.

OBB: an oriented bounding box (OBB), which is a quite well-known bounding box type. The OBB of a given object is defined as including the object and any minimum cuboid relative to a coordinate axis direction.

SPEE: Spee Curve, which is a connecting line for connecting the incisal edge of a mandibular incisor, a dental cusp of a cuspid tooth, the buccal tip of a premolar, and proximal and distal buccal tips of molars. The connecting line is a concavely upward curve from front to back, which is also referred to as the Spee curve. An incisor segment of the curve is relatively straight, the curve gradually decreases backwards from the cuspid tooth to the distal buccal tips of the premolar and the first molar, and then gradually increases from the buccal tips of the second and third molars.

CGAL: an abbreviation of Computational Geometry Algorithms Library, which provide an efficient and reliable algorithm library by using a C++ language.

Ceres Solver: an open source library, which is an efficient and convenient tool for solving many nonlinear optimization problems.

In order to solve the problems mentioned in the background art, according to one aspect of the embodiments of the present disclosure, an embodiment of a gum line extraction method is provided.

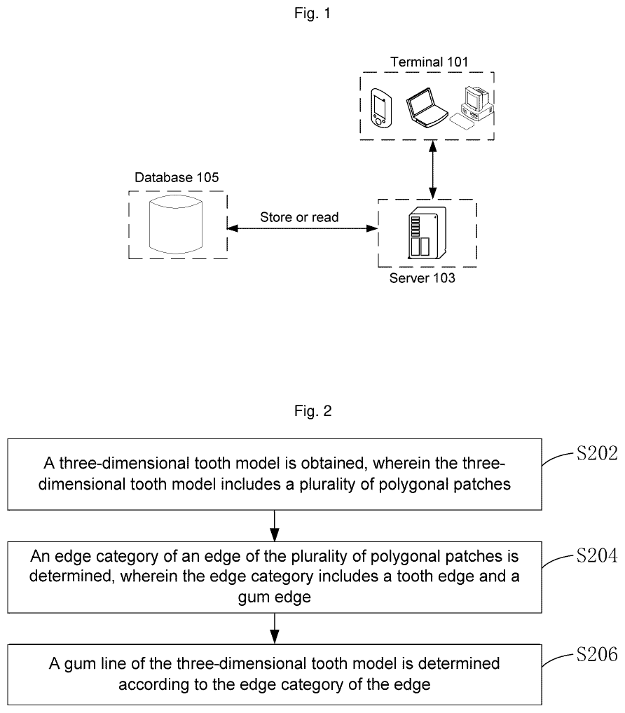

Optionally, in the embodiments of the present disclosure, the gum line extraction method may be applied to a hardware environment composed of a terminal 101 and a server 103 as shown in FIG. 1. As shown in FIG. 1, the server 103 is connected with the terminal 101 by a network and may be configured to provide services for the terminal or a client installed on the terminal, a database 105 may be provided on the server or independent of the server, and is configured to provide a data storage service for the server 103, the network includes, but is not limited to, a wide area network, a metropolitan area network or a local area network, and the terminal 101 may be an apparatus, configured to acquire a three-dimensional tooth model, such as an oral cavity scanning model scanner, or a terminal, configured to receive the three-dimensional tooth model, such as a mobile phone or a computer, etc.

The gum line extraction method in the embodiments of the present disclosure may be executed by the server 103, and may also be jointly executed by the server 103 and the terminal 101.

As shown in FIG. 2, the method may include the following steps:

Step S202, a three-dimensional tooth model is obtained, wherein the three-dimensional tooth model includes a plurality of polygonal patches;

Step S204, an edge category of an edge of the plurality of polygonal patches is determined, wherein the edge category includes a tooth edge and a gum edge; and

Step S206, a gum line of the three-dimensional tooth model is determined according to the edge category of the edge.

Optionally, based on the gum line extraction method in the present embodiment, the gum line of the three-dimensional tooth model of teeth of a user may be determined, so that subsequent tooth segmentation, dental device cutting operation and the like may be performed according to the gum line.

The three-dimensional tooth model may be an oral cavity scanning model, and the oral cavity scanning model refers to a digitized three-dimensional model that is generated by scanning the interior of the oral cavity of the user and includes tooth parts and gum parts of the user; and of course, the three-dimensional tooth model may also be a digitized three-dimensional model that is obtained by firstly acquiring impressions of the tooth parts and the gum parts in an impression extraction manner and then scanning the impressions. The data of the three-dimensional tooth model may be configured to display the shape or style of the three-dimensional tooth model on a computer or a server by a display screen for the convenience of viewing of a doctor.

The polygonal patch is a patch that forms the surface (tooth parts and non-tooth parts, the surface of the entire model) of the three-dimensional tooth model, and may be in the shape of a triangle, a quadrangle, a pentagon . . . , and the like. Each polygonal patch is located on a plane, and different polygonal patches may be located on the same plane or different planes. The surface of the three-dimensional tooth model is composed of a plurality of polygonal patches, and each polygonal patch includes a plurality of edges, and two adjacent polygonal patches share one edge. Endpoints of the edges are vertices of the corresponding polygonal patches, and one vertex may be shared by the plurality of polygonal patches.

It should be noted that the three-dimensional tooth model includes tooth parts and gum parts, and the gum line is a boundary between the tooth parts and the gum parts. The edges forming the polygonal patches may be located on the tooth parts or the gum parts, and thus can be classified into tooth edges and gum edges. On this basis, in the present embodiment, the gum line of the three-dimensional tooth model may be determined according to the edge categories of the edges of the polygonal patches, and the determined gum line is configured to segment the tooth parts and the gum parts of the three-dimensional tooth model.

After the gum line is obtained, a tooth film may be cut on a position of the three-dimensional tooth model according to the position of the gum line, to obtain a dental device, such as an invisible appliance, or the gum line is configured to perform tooth segmentation on the three-dimensional tooth model.

In the present embodiment, after the three-dimensional tooth model is obtained, the categories of the edges may be determined by a method for determining the edge categories of the edges of the polygonal patches on the three-dimensional tooth model, then the gum line of the three-dimensional tooth model is determined, and the edges, serving as components of the polygonal patches, may further refine the three-dimensional tooth model, so that the accuracy of extracting the gum line of the tooth model can be improved.

As an optional example, determining the edge categories of the edges of the plurality of polygonal patches includes: obtaining target features of the edges of the plurality of polygonal patches; and determining the edge categories of the edges of the plurality of polygonal patches according to the target features.

In the present embodiment, the target features include geometric features, of course, in other embodiment, the target features may include non-geometric features, which is not specifically limited herein. In the present embodiment, the edge categories may be determined by a method for extracting the target features of the edges of the polygonal patches and identifying the target features. It should be noted that, when the target features include the geometric features, a topological structure relationship of a space is considered when edge classification is performed, thereby improving the accuracy of identifying the gum line.

In the present embodiment, the target features of all edges on the three-dimensional tooth model may be extracted, and the target features of some of the edges may also be selected. The edge categories of the edges are determined by identifying the extracted target features of the edges.

As an optional example, the target feature of an edge includes at least one of the following: a dihedral angle between a first polygonal patch and a second polygonal patch that share an edge, a first curvature value of a first vertex of the edge, a second curvature value of a second vertex of the edge, a first distance to an edge from a vertex of the first polygonal patch that is away from the edge, a second distance to an edge from a vertex of the second polygonal patch that is away from the edge, a first angle opposite to a edge of the first polygonal patch, and a second angle opposite to a edge of the second polygonal patch. In one application scenario, the target feature of the edge includes the foregoing seven items.

As another optional example, the target feature of an edge may include at least two of the following features: a dihedral angle between a first polygonal patch and a second polygonal patch that share the edge, a first curvature value of a first vertex of the edge, a second curvature value of a second vertex of the edge, a first spatial coordinate of the first vertex, a second spatial coordinate of the second vertex, the length of the edge, an included angle between the edge and an adjacent edge, a first distance to the edge from a vertex of the first polygonal patch that is away from the edge, a second distance to the edge from a vertex of the second polygonal patch that is away from the edge, a first angle opposite to a edge of the first polygonal patch, a second angle opposite to a edge of the second polygonal patch, a first normal of the first vertex, a second normal of the second vertex, a normal of the first polygonal patch, and a normal of the second polygonal patch.

The dihedral angle between the first polygonal patch and the second polygonal patch that share the edge is an included angle between the two polygonal patches that share one edge. The first vertex and the second vertex are two endpoints of the edge, and the curvature value is a value of the curvature of the patch corresponding to the vertex at the vertex. The curvature is a rotation rate of a tangential direction angle of a certain point on the curve with respect to an arc length, which is defined by differentiation, indicates the degree of the curve deviating from a straight line and indicates a numerical value of the degree of bending of the curve at a certain point. The greater the curvature is, the more bent the curve where a corresponding point is located is at the point. The spatial coordinate is a coordinate in a three-dimensional rectangular coordinate system where the vertex is located. The three-dimensional rectangular coordinate system may be a coordinate system where the three-dimensional tooth model is located, for example, a plane where a bottom plate of the three-dimensional tooth model is located may be defined as a xOy plane, and the normal direction of the plane is defined as a Z axis. The direction of the coordinate axis may be predetermined. For example, a gum plane of the three-dimensional tooth model is used as a plane where an X axis and a Y axis are located, and the tooth direction is used as a

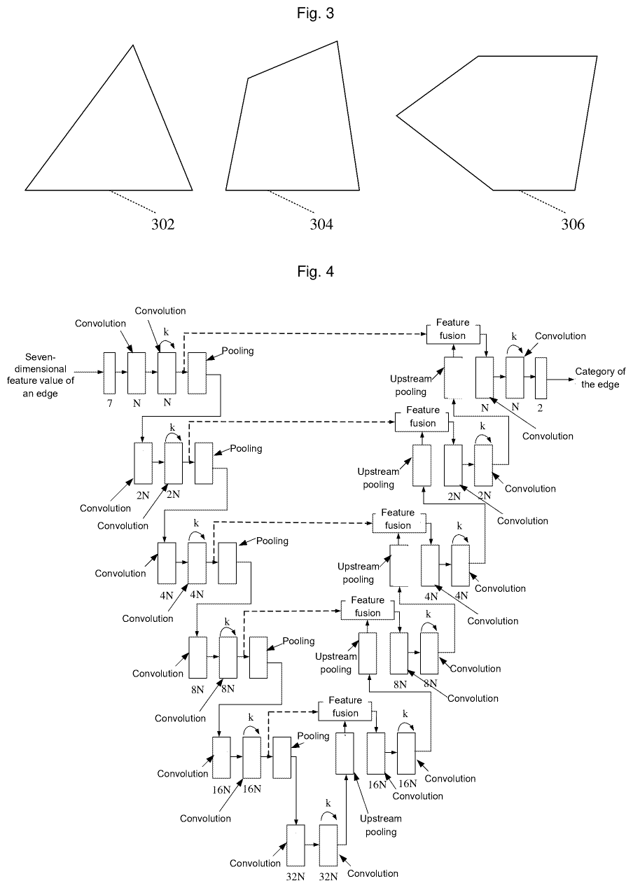

Z-axis direction. The length of the edge is the distance between the two endpoints of the edge; since the edge may have a plurality of adjacent sides, there may be a plurality of included angles between the edge and the adjacent edges, and the included angles range from 0 degree to 180 degrees; when the polygon is quadrilateral or more, there may be more than one distance to the edge from the vertex that is away from the edge, the distance from each vertex to the edge may be different, and when the polygonal patch is a triangular patch, there is only one vertex away from the edge, therefore there is only one distance from the vertex to the edge; and similarly, the edge may have one or more opposite angles in the polygonal patch, and when there are a plurality of opposite angles, the degree of each opposite angle may be different. For example, as shown in FIG. 3, the polygonal patches are respectively a triangular patch 302, a quadrilateral patch 304 and a pentagonal patch 306, and each edge of the three polygonal patches corresponds to at least one or at least two of the above features.

For example, during feature extraction, the polygonal patches corresponding to the edges of the three-dimensional tooth model are found; the included angle between the normals of two polygonal patches may be obtained according to the normals of the polygonal patches by means of multiplication cross, the included angles between the dihedral angles of the two polygonal patches and the normals are complementary, and thus the dihedral angles of the two polygonal patches may be obtained by subtracting the included angles between the normals from 180 degrees; the curvature values of two vertexes may be obtained according to a curvature geometric calculation method or other methods; the distance to the edge from the vertex of the polygonal patch that is away from the edge may obtained according to the distance from a point to a straight line; and two vectors are obtained according to the orientation of two edges, and the included angle between the two edges at the vertex of the polygonal patch may be obtained according to the two vectors by means of point multiplication, that is, the opposite angle of the edge in each polygonal patch is obtained. It should be noted that the spatial coordinate corresponding to each vertex and the neighborhood relationship between the points, the edges and the surfaces are known, and thus each target feature may be obtained on this basis.

As an optional example, determining the edge category of the edge according to the target feature includes: performing dimension raising on the target feature with an initial dimension of the edge to obtain a first feature of a first dimension; performing dimension reduction on the first feature to obtain a second feature with a target dimension; and determining that the edge is a tooth edge or a gum edge according to the magnitude of the value of the second feature. That is, in the present embodiment, the target dimension may be two dimensions, which correspond to the tooth edge and the gum edge, respectively. Certainly, in other embodiments, it may be multi-dimensional, as long as the edge category of the corresponding edge can be determined, which is not limited herein.

Optionally, in the present embodiment, after the target feature of the edge is extracted, the initial dimension of the target feature may be determined according to the type, the number and the like of the extracted target feature. dimension raising is firstly performed on the target feature to increase the same to the first dimension, to obtain the first feature, and then dimension reduction is performed on the first feature to reduce the same to the target dimension, however, since the dimension raising is firstly performed and then the dimension reduction is performed, the first feature that has been subjected to dimension reduction is no longer the target feature, but the second feature of the target dimension, so that the edge category of the corresponding edge may be obtained accordingly.

Taking it as an example that the initial dimension is 7 dimensions, a seven-dimensional target feature of a corresponding edge is input into an edge classification network to classify the corresponding edge. For example, firstly, an input dimension is 7 dimensions, which respectively correspond to 7 geometric features, the dimension of the target feature is raised to N dimensions by means of one convolution operation, then the dimension of the target feature is kept unchanged by means of k identical convolution operations, and the number of edges is reduced by a pooling operation. Then, the above process is repeated, that is, one dimension-raising convolution+k identical-dimensional convolutions+pooling, so that the dimension of the target feature becomes 2N, 4N and 8N in sequence. It should be noted that in the last dimension-raising cycle, an upstream pooling operation is performed after the k identical-dimensional convolutions to gradually recover the number of the edges, the dimension-reduction convolution is performed to reduce the dimension of the feature to 4N dimensions, the dimension of the feature is kept unchanged by means of k identical convolution operations, and then the above process is repeated, that is, upstream pooling+one dimension-reduction convolution+k dimension-raising convolutions, so that the dimension of the target feature becomes 4N, 2N and N in sequence. It should be noted that after the last dimension-reduction cycle, the dimension of the feature is further directly reduced to 2 dimensions. In FIG. 4, a dotted arrow represents a feature fusion operation, the final values of the two dimensions are probability values of the edge being the tooth edge and the gum edge, the category with a large probability value may be selected as the category of the edge, and so far, each edge of the model is classified as the tooth edge or the game edge.

It should be noted that, the above description is merely an example and is not a limitation. For example, the initial dimension is not limited to 7 dimensions, the number of cycles during the dimension raising and dimension reduction processes is not limited neither, N is an integer not less than 7, for example, 7, 8, 9, 10, etc., and in a specific implementation solution, N may be adjusted according to actual situations.

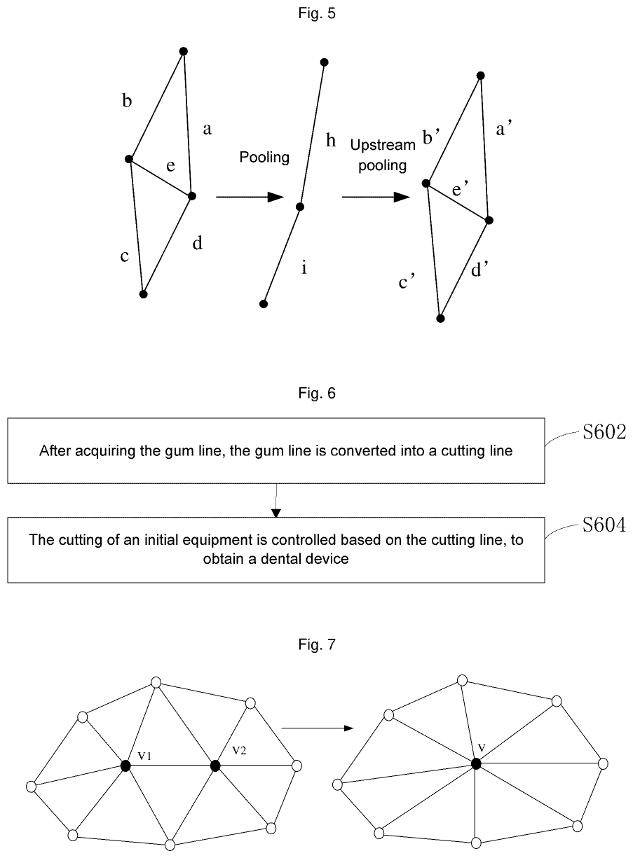

It should be further noted that convolution and pooling in the step are different from convolution and pooling operations in an image neighborhood, and as shown in FIG. 5, the operation thereof is performed based on the edges in the three-dimensional tooth model.

In order to eliminate the influence caused by different edge sequences (e.g., (a, b, c, d) and (c, d, a, b) in the convolution operation, an original edge feature is processed according to the following formula (1), wherein f(a) and f′(a) respectively represent an original feature and a processed feature of an edge a, and the convolution operation of a processed edge e is defined as formula (2), wherein {Wₖ|k=0,1,2,3,4} represents a weight parameter to be trained. The pooling operation converts 5 edges (e, a, b, c, d) into 2 edges (h, i), the definition of which is shown in formula (3), and the upstream pooling operation is opposite to the pooling operation, that is, the 2 edges (h, i) are converted into (e′, a′, b′, c′, c′), and the definition of which is shown in formula (4)

$${f}^{′} ( a ) = \vert f ( a ) - f ( c ) \vert \qquad ( 1 )$$

$${f}^{′} ( b ) = f ( a ) + f ( c )$$

$${f}^{′} ( c ) = \vert f ( b ) - f ( d ) \vert$$

$${f}^{′} ( d ) = f ( b ) + f ( d )$$

$$C ( e ) = {w}_{0} {{f}^{′}}^{} ( e ) + {w}_{1} {f}^{′} ( a ) + {w}_{2} {f}^{′} ( b ) + {w}_{3} {f}^{′} ( c ) + {w}_{4} {f}^{′} ( d ) \qquad ( 2 )$$

$$f ( h ) = \frac{1}{3} ( f ( e ) + f ( a ) + f ( b ) ) \qquad ( 3 )$$

$$f ( i ) = \frac{1}{3} ( f ( e ) + f ( c ) + f ( d ) )$$

$$f ( {e}^{′} ) = \frac{1}{2} ( f ( h ) + f ( i ) ) \qquad ( 4 )$$

$$f ( {a}^{′} ) = f ( {b}^{′} ) = f ( h )$$

$$f ( {c}^{′} ) = f ( {d}^{′} ) = f ( i )$$

In addition, before edge classification is performed, the classification network needs to be 5 trained first to obtain a network weight value and values of some hyper-parameters, for example, k, N, or the like. For example, a plurality of groups of geometric feature data and category values (for example, a gum corresponds to 0 and a tooth corresponds to 1) of classification of corresponding edges are input into the edge classification network for training, for example, 500 groups, 1000 groups or 2000 groups of classified tooth model data are input, and each group of 10 tooth model data includes geometric feature data and classification data corresponding to 5000, 10000 or 20000 edges, so that the classification network can classify the edges according to geometric feature data of the edges. It should be noted that in the present embodiment, gum line data may be automatically identified in combination with the deep learning technology and based on an edge classification method, so that the identification accuracy and efficiency can be greatly improved, the efficiency of the entire dental diagnosis and treatment process, such as invisible orthodontics, is improved, and the user experience in diagnosis and treatment is enhanced.

As an optional example, determining the gum line of the three-dimensional tooth model according to the edge categories of the edges includes: determining patch categories of the polygonal patches according to the edge categories, wherein the patch categories include tooth patches and gum patches; and extracting the gum line based on shared edges of adjacent polygonal patches of different patch categories.

Optionally, in the present embodiment, after the categories of the edges are determined, the patch category of the polygonal patch may be determined based on the categories of the edges. It can be understood that the tooth patch is a patch located on the tooth parts of the tooth model, and the gum patch is a patch located on the gum part. The patch category is determined according to the edge categories of the edges, and the patch category of the polygonal patch may be determined according to all edges of the polygonal patch, or the patch category of the polygonal patch may be determined according to at least one edge of the polygonal patch.

As an optional example, determining the patch category of the polygonal patch according to the edge category includes: when the number of gum edges is greater than the number of tooth edges in the polygonal patch, determining that the polygonal patch is a gum patch; and when the number of gum edges is less than the number of tooth edges in the polygonal patch, determining that the polygonal patch is a tooth patch.

Optionally, in the present embodiment, the patch category of the polygonal patch is determined according to the edge categories of all edges of the polygonal patch. According to the number of the tooth edges and the number of the gum edges in the polygonal patch, it is determined that the polygonal patch is a tooth patch or gum patch.

All edges of the three-dimensional tooth model are traversed, and if two patch categories corresponding to an edge are different, the edge is marked as a shared edge.

As an optional example, extracting the gum line based on the shared edges of the adjacent polygonal patches of different patch categories includes: determining first feature points based on vertexes of the shared edges; determining target feature point from the first feature points according to a curvature relationship; and determining the gum line according to the target feature point.

Optionally, in the present embodiment, since the patch categories of the polygonal patches may be determined according to the edge categories of the edges, after the patch categories are determined, the shared edges of the polygonal patches of different categories are the boundaries of the tooth patches and the gum patches. Thus, the gum line may be determined according to all shared edges of the tooth patches and the gum patches.

In the present embodiment, the first feature points may be determined from the vertexes of the shared edges, then the target feature point is determined from the first feature points, and the gum line is determined according to the target feature point. The target feature points may be calculated based on a curvature.

In the present embodiment, the vertex of one shared edge may be used as a starting point to search for a connected shared edge, and the vertexes of the connected shared edges are recorded in a counterclockwise or clockwise sequence to serve as feature points, wherein the first feature points may be all or part of the recorded feature points, and specifically, the first feature points are located on an initial tooth model, so that the accuracy of the extracted gum line is higher. The target feature points are a part of feature points having specific features in the first feature points.

As an optional example, determining the first feature point based on the vertex of the shared edge includes: in a case where the three-dimensional tooth model is a model obtained by performing edge contraction processing on the initial tooth model, determining a point closest to the vertex of the shared edge on the initial tooth model as the first feature point; and in a case where the three-dimensional tooth model is the initial tooth model, determining the vertex on the shared edge as the first feature point.

Optionally, the initial tooth model is an initially acquired tooth model. The edge contraction processing may be performed on the initial tooth model to obtain the three-dimensional tooth model, or the initial tooth model is directly used as the three-dimensional tooth model. Whether to perform the edge contraction processing on the initial tooth model affects whether to directly use the vertex of the shared edge as the first feature point. In the present embodiment, the first feature point is the vertex of the shared edge, or the point closest to the shared edge on the initial tooth model.

That is to say, in the present embodiment, after the vertex of one shared edge is used as the starting point to search for the connected shared edges, and then the vertexes are recorded in the counterclockwise or clockwise sequence to serve as the feature points, the recorded feature points may be projected back to an original model, and points closest to the recorded feature points on the original model are found as new feature points.

For example, a KDTree may be established via the vertexes of the initial tooth model, the KDTree is a tree-like data structure that stores instance points in a k-dimensional space for fast retrieval. In the present embodiment, all recorded feature points may be traversed, and points closest to the feature points may be searched for in the KDTree to serve as the new feature points.

The new feature points are the first feature points.

As an optional example, determining the target feature point from the first feature points according to the curvature relationship includes: determining, as the target feature point, a first feature point whose curvature is greater than the curvature of an adjacent first feature point in the first feature points.

After the first feature points are determined according to whether the initial tooth model has been subjected to edge contraction processing, the target feature point may be determined from the first feature points according to the curvatures of the first feature points. Each first feature point corresponds to one curvature. In the present embodiment, the first feature point whose curvature is greater than the curvature of the adjacent first feature point in the first feature points is used as the target feature point, that is, the point where the located position is more bent in the curve is determined as the target feature point, so that the actual trend of the curve can be better reflected.

After the recorded feature points are projected back to the original model, the curvatures of discrete points may be estimated by using a geometric algorithm of curvatures, and a feature point with a large curvature is reserved as the target feature point; and the reason for reserving the feature point with the large curvature is that the point with the large curvature is usually a turning point, and the feature of the turning point is relatively strong and is substitutive.

As an optional example, determining the gum line according to the target feature points includes: performing an interpolation operation on the target feature point to obtain interpolated target feature points; and connecting the interpolated target feature points in sequence to form the gum line.

In the present embodiment, by performing the interpolation operation on the target feature point, the loss of the feature points caused by determining the target feature point from the first feature points can be compensated, and the target feature point may be combined into a closed curve by the interpolation operation. The closed curve is the gum line.

When interpolation is performed on the target feature point, B spline interpolation may be performed on adjacent target feature points; all points are connected in sequence, and the formed curve is the gum line. After the gum line is obtained, a film may be cut based on the gum line to obtain an invisible appliance or to perform tooth segmentation on the three-dimensional tooth model. It should be noted that when interpolation is performed on the target feature point, it is not limited to a B spline, and other splines may also be used, which is not specifically limited herein.

As an optional example, obtaining the three-dimensional tooth model includes: acquiring an initial tooth model, wherein the initial tooth model includes a plurality of polygonal patches, and the number of edges in the initial tooth model is greater than the number of edges in the three-dimensional tooth model; and reducing the number of edges in the initial tooth model to a preset number in a preset mode, to obtain the three-dimensional tooth model.

Optionally, in the present embodiment, the preset mode may be an edge contraction operation or an edge collapse operation. The purpose of performing the edge contraction operation on the initial tooth model is to reduce the number of edges in the initial tooth model, to obtain the three-dimensional tooth model to simplify the tooth model and to improve the identification rate of the gum line.

There may be a plurality of methods to reduce the number of edges of the initial tooth model. One edge or a batch of edges may be reduced, for example, one edge is reduced every time until the edges are reduced to the preset number or for a preset number of times. Or, a batch of edges is reduced every time until the edges are reduced to the preset number or for the preset number of times.

Taking the edge collapse operation as an example, the imported initial tooth model may be a tooth model in any direction, and the initial tooth model is a digitized three-dimensional body composed of a series of vertexes and polygonal patches. After the initial tooth model is imported, down sampling is performed on the tooth model in an edge collapse mode, the polygonal patches of the tooth model are sparser after the down sampling, since the number of polygonal patches is reduced, the number of edges is also reduced, and the down sampling is stopped until there is a specified number of edges.

The imported initial tooth model usually has a plurality of edges and the number of edges is inconsistent, and when classification is performed in subsequent steps by using the classification network, the number of required edges is often specific, so that the down sampling needs to be performed on the imported initial tooth model to delete some edges and to reverse the specified number of edges. When the number of edges is determined, a classification speed (the smaller the number of edges is, the higher the speed is) and the accuracy of the finally obtained gum line (the greater the number of edges is, the higher the accuracy is, and the greater the number of edges is, the smaller the information loss of the original model is) may be taken into consideration. The main flows are as follows:
(1) The sum of distances from the vertexes of each edge of the initial tooth model to adjacent patches is recorded as an error value, an initial value of which is 0, wherein the adjacent patches refer to patches where the vertexes are located, and the initial error value being 0 means that when edge contraction is not performed, the sum of the distances from the vertexes to the adjacent patches is 0.
(2) Error values (here refer to the sums of the distances from the vertexes to the adjacent patches when edges are not deleted) corresponding to the vertexes after edges are deleted are calculated, the error values are sorted from small to large, and the edges with small errors are firstly deleted, wherein when one edge is contracted to one vertex, two adjacent patches are deleted, and one vertex and three edges are deleted; there are a plurality of cases for the position of contracting one edge to a point, for example, the two vertexes of the edge may be moved to any point on the edge for deletion, and the specific position of the point is selected according to a minimum error value in the step.
(3) The above steps are repeated to continuously calculate the error values and to delete the corresponding edges until the number of edges reaches the specified number of edges. So far, the adjustment on the number of edges of the initial tooth model is completed.

As an optional example, reducing the number of edges in the initial tooth model to the preset number in the preset mode, to obtain the three-dimensional tooth model includes: for each edge of a tooth model to be processed, calculating the sum of distances from vertexes corresponding to each edge to adjacent polygonal patches of the vertexes corresponding to each edge in the tooth model to be processed after edge contraction processing is performed on each edge according to each of a plurality of contraction modes, wherein during the first-time processing, the tooth model to be processed is the initial tooth model; determining the edge and the contraction mode corresponding to a minimum value among a plurality of distance sums obtained by calculation; and performing the edge contraction processing on the edge corresponding to the minimum value according to the contraction mode corresponding to the minimum value, to obtain the three-dimensional tooth model.

As an optional example, performing the edge contraction processing on the edge corresponding to the minimum value according to the contraction mode corresponding to the minimum value, to obtain the three-dimensional tooth model includes: after the edge contraction processing is performed, if the number of edges in the obtained tooth model is not greater than the preset number, the obtained tooth model is the three-dimensional tooth model; and if the number of edges is greater than the preset number, determining the obtained tooth model as the tooth model to be processed, so as to continue to perform the edge contraction processing.

Optionally, in the present embodiment, when the edge contraction processing is performed on the initial tooth model, it is also necessary to determine to contract which edge. This process may be a circulating process. That is, one edge is contracted every time and circulation is performed or a batch of edges is contracted every time and circulation is performed, until the contracted tooth model to be processed meets the condition. Thus, during every contraction, it is necessary to determine to contract which edge or which batch of edges.

In the present embodiment, the initial model may be used as the tooth model to be processed, and the edge to be contracted is determined for the tooth model to be processed. The determination method may be to firstly perform simulation or calculation, if a certain edge is contracted, the sum of distances from vertexes corresponding to each edge in the contracted model to adjacent polygonal patches of the vertexes corresponding to the edge in the tooth model to be processed is calculated, if the sum is the minimum, then the edge is contracted according to the strategy. The calculation is repeated every time when the contraction is performed, and the edge contraction is repeated until the number of edges of the tooth model to be processed meets the requirement of the preset number. So far, the edge contraction is completed, and the three-dimensional tooth model is obtained.

According to another aspect of the embodiments of the present disclosure, further provided is a manufacturing method of a dental device, as shown in FIG. 6, including:

S602, after acquiring the gum line according to the above gum line extraction method, the gum line is converted into a cutting line; and

S604, the cutting of an initial equipment is controlled based on the cutting line, to obtain the dental device.

With regard to the gum line extraction method, reference is specifically made to the foregoing embodiments, and thus details are not repeated here again. It should be noted that in the present embodiment, the initial equipment has an association relationship with the three-dimensional tooth model. For example, the initial equipment may be generated based on the initial tooth model or the three-dimensional tooth model.

Optionally, in the present embodiment, after the gum line is determined, the initial equipment may be obtaining by cutting the three-dimensional tooth model according to the position of the gum line, the initial equipment is a shell-shaped film surrounding a solid tooth model, and the solid tooth model is formed based on the initial tooth model or the three-dimensional tooth model in 3D printing or other manners, so that the shape of the side of the shell-shaped diaphragm that is subjected to film pressing is the same as the structure of the tooth parts of the solid dental model, or the tooth parts have the same structure as at least part of the gum parts. By cutting, the target equipment the same to the part of surrounding the tooth parts of the three-dimensional tooth model may be cut from the initial equipment, that is, a tooth film, which may be specifically an invisible appliance.

As an optional example, in a case where an initial equipment to be cut is acquired, before obtaining the initial equipment by cutting the three-dimensional tooth model according to a first position of the gum line, the gum line extraction method further includes: performing at least one of smoothing processing and position adjustment processing on the gum line.

In the present embodiment, before the initial equipment is obtained by cutting according to the gum line, or before the gum line is converted into the cutting line, the gum line may also be adjusted. The adjustment includes the smoothing process and the position adjustment process, wherein the smoothing process refers to that after a final gum line is generated, the gum line is tightly attached to a real gum line, and the intersection between one tooth and another tooth is relatively sharp, which does not meet generation process requirements, therefore the gum line needs to be flattened at the position, that is, an acute angle is removed from the gum line. Similarly, the position adjustment processing refers to that after the gum line is generated, the gum line is tightly attached to the real gum line, which does not meet the process requirements after generation, therefore a certain margin needs to be reserved, that is, the gum line is offset downwards (or offset upwards) on the whole. The degree of the acute angle in the acute angle removal processing is defined to be less than 180°, and the overall offset of the offset processing ranges from 0 mm and 2 mm. The gum line that has been subjected to the post processing may be used as a cutting curve.

The gum line extraction method in the present disclosure may be mainly applied to a correction presentation scenario, in which after the gum line corresponding to the tooth model of the user is determined, tooth segmentation may be performed according to the gum line, or the film is obtaining by cutting according to the gum line. The design of an orthodontic guide plate, the design of a substitutive tooth model, and the design of temporary teeth may be further performed based on tooth segmentation data.

In the present embodiment, if the teeth of the user are to be checked or corrected, the oral cavity of the user may be scanned at first to obtain the data of the three-dimensional tooth model of the teeth of the user. The data may be displayed to the user and the doctor by the display screen, so that the doctor can introduce the situations of the teeth for the user.

The three-dimensional tooth model of the user obtained by oral cavity scanning may be a closed or non-closed model. The three-dimensional tooth model includes a tooth regions and gum regions, and the boundaries between the tooth regions and the gum regions are not labeled. Thus, if tooth segmentation needs to be performed, a segmentation line (the gum line) between teeth and gums needs to be determined.

For example, firstly, an imported 3D tooth model, that is, the initial tooth model, is acquired, after the initial tooth model is imported, down-sampling processing is performed on the initial tooth model to reduce the number of edges of the initial tooth model, to obtain a tooth model with a specified number of edges, the down-sampling effect may refer to FIG. 7, two points V1 and V2 are down-sampled to V, and then the number of edges of the initial tooth model is reduced. The target feature of each edge of the initial tooth model is calculated, the category of the edge is obtained according to the feature of the edge via the edge classification network, the edge is divided into two categories, that is, a tooth edge and a gum edge, the category with a greater value is selected from a prediction result to serve as the category of the edge, and the structure of the edge classification network is shown in FIG. 4. Boundary vertexes for segmenting the gums from the teeth are obtained by using classification information of the edges, these vertexes are used as feature points; these feature points are projected back to the initial tooth model, and the closest points on the initial tooth model are found to serve as first feature points on the initial tooth model; feature points having greater curvatures in the first feature points are reserved as target feature points; and interpolation is performed on the target feature point on the initial tooth model to obtain a closed curve, that is, the gum line. The automatic gum line identification method of the tooth model greatly promotes the automatic generation and processing flow of tooth correction, and improves the working efficiency and the user experience.

According to another aspect of the embodiments of the present disclosure, further provided is a manufacturing method of a dental device, wherein the manufacturing method of the dental device may include the following steps:

Step 1: a digitized tooth model is acquired.

The tooth model may be the three-dimensional tooth model in the foregoing gum line extraction method embodiments. Correspondingly, the acquisition mode of the tooth model may be consistent with the acquisition mode of the three-dimensional tooth model, that is, the tooth model may be acquired in the oral cavity scanning mode, and may also be acquired in the traditional impression extraction mode, which is not limited herein.

Step 2: pre-processing is performed on the digitized tooth model.

In one embodiment, the pre-processing operation may include the steps in the gum line extraction method to identify a gum line, and may further include converting the gum line into a cutting line, so that an initial equipment is obtained by cutting in subsequent steps to obtain a dental device for use, or in some application scenarios, the gum line may also be applied to automatic tooth segmentation, gum and dental crown separation, and the like. The gum line extraction method may be the same as that in the foregoing gum line extraction method embodiments, therefore for related detailed content, reference is made to the foregoing content, and thus details are not described herein again.

In another embodiment, the pre-processing operation may further include performing a tooth segmentation operation on the three-dimensional tooth model.

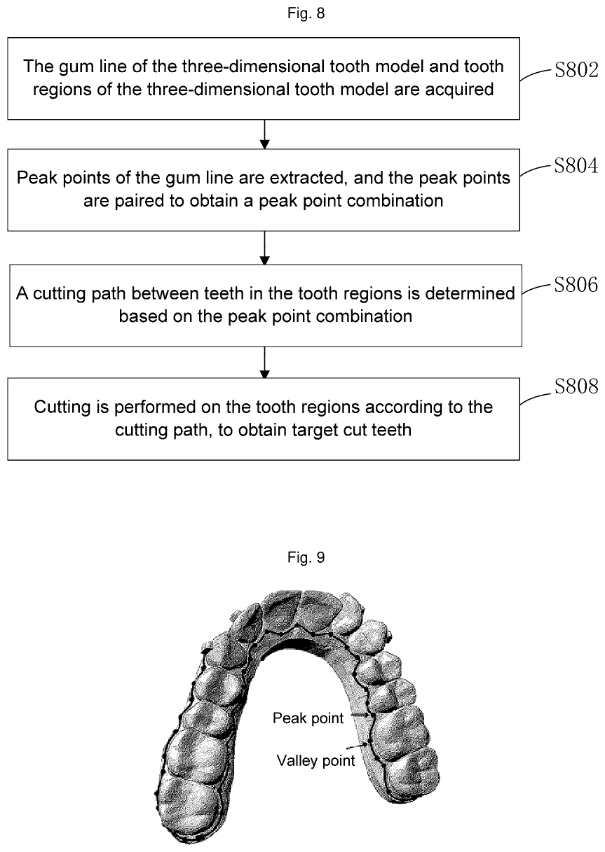

In some application scenarios, the tooth segmentation operation may be performed based on the extracted gum line. For example, as shown in FIG. 8, the method for performing the tooth segmentation operation on the three-dimensional tooth model may include the following steps:

Step S802, the gum line of the three-dimensional tooth model and tooth regions of the three-dimensional tooth model are obtained;

Step S804, peak points of the gum line are extracted, and the peak points are paired to obtain a peak point combination;

Step S806, a cutting path between teeth in the tooth regions is determined based on the peak point combination; and

Step S808, Cutting is performed on the tooth regions according to the cutting path, to obtain target cut teeth.

It should be noted that the three-dimensional tooth model herein may be understood as the foregoing three-dimensional tooth model.

Optionally, the above three-dimensional tooth model segmentation method aims to accurately divide each tooth on the three-dimensional tooth model into single teeth.

In the present disclosure scenario, the acquisition mode of the gum line may be same as that in the foregoing gum line extraction implementation, therefore for related detailed content, reference may be made to the foregoing content, and thus details are not described herein again. Certainly, the gum line may also be determined by identifying a two-dimensional projection image of the three-dimensional tooth model, which is not limited herein.

Optionally, the peak point of the three-dimensional tooth model may be a point higher than an adjacent point on the gum line of the three-dimensional tooth model. For example, on the gum line of the three-dimensional tooth model, a straight line formed by connecting a plurality of points continues to go high, and when one point starts to descend, then the point is a peak point. The peak point may be understood as the highest point of the gum located at a slit between two teeth.

By means of pairing the peak points in the present embodiment, every two peak points may be determined as a peak point combination, and the two peak points in one peak point combination are configured to determine the boundary between two teeth. Each peak point combination includes two peak points.

In the present embodiment, after the peak point combination is determined, a segmentation path between two teeth in each tooth region may be determined according to the two peak points.

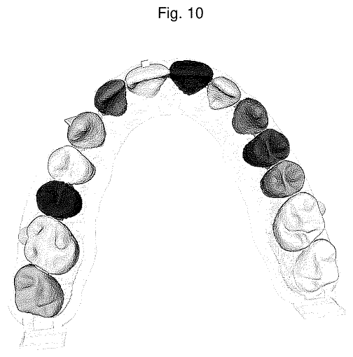

The tooth regions may be segmented according to the segmentation path to obtain single teeth. As shown in FIG. 9, there are peak points and valley points on the gum line between the teeth and the gums of the three-dimensional tooth model (there are also peak points and valley points on a non-displayed side of the teeth in FIG. 9), the peak point is a higher point (which may be the highest point or not the highest point, and is located on the gum line) of the gum part located between the two teeth on the gum, and the valley point is a lower point on the gum line. The peak points are paired, the peak points at the slit position between every two teeth may be paired as one peak point combination, the segmentation path between the two teeth may be determined by the peak point combination, and finally the teeth are segmented by the segmentation path to obtain single teeth. The segmentation result may be shown in FIG. 10.

In the present disclosure, by means of the method for acquiring the gum line of the three-dimensional tooth model and the tooth regions of the three-dimensional tooth model, extracting the peak points of the gum line, and pairing the peak points to obtain the peak point combination, then determining the segmentation path according to the peak point combination, and performing segmentation processing on the tooth regions according to the segmentation path, to obtain the target segmented teeth, each tooth of the three-dimensional tooth model can be accurately segmented, thereby achieving the effect of accurately performing tooth segmentation on the three-dimensional tooth model.

As an optional example, acquiring the tooth regions of the three-dimensional tooth model includes: performing segmentation processing on the three-dimensional tooth model based on the gum line, to obtain the tooth regions of the three-dimensional tooth model.

Optionally, in the present embodiment, before tooth segmentation is performed on the three-dimensional tooth model, segmentation processing may be firstly performed on the three-dimensional tooth model by using the gum line, to obtain the tooth regions of the three-dimensional tooth model after the segmentation is completed. The segmentation of the three-dimensional tooth model may be regarded as hiding or not processing the gum region of the three-dimensional tooth model, and when tooth segmentation is subsequently performed on the three-dimensional tooth model, the influence of the gum regions on the tooth segmentation precision may be avoided.

As an optional example, before acquiring the gum line of the three-dimensional tooth model, the method further includes: determining an initial orientation of the three-dimensional tooth model; and adjusting the three-dimensional tooth model from the initial orientation to a target orientation.

Optionally, in the present embodiment, after the oral cavity scanning data of the user is acquired to obtain the three-dimensional tooth model of the user, the direction of the three-dimensional tooth model may be adjusted. The purpose of adjusting the three-dimensional tooth model is to “straighten” the three-dimensional tooth model, so that all three-dimensional tooth models can be adjusted to a fixed orientation, and thus the curvature identification is more accurate. For example, for all three-dimensional tooth models, the initial orientations may be different. If a table top is used as horizontal plane, the direction of an upward normal of the table top may be used as the target orientation. Then, after the three-dimensional tooth model is adjusted to the target orientation, the three-dimensional tooth model may be in a tooth upward state. After the three-dimensional tooth model is obtained, the orientation of the three-dimensional tooth model may be adjusted to the target orientation, so that the directions of all acquired three-dimensional tooth models can be unified.

As an optional example, determining the initial orientation of the three-dimensional tooth model includes: in a case where the three-dimensional tooth model is a non-closed model, determining a minimum oriented bounding box of the three-dimensional tooth model; and determining the initial orientation of the three-dimensional tooth model according to the minimum oriented bounding box.

Optionally, in the present embodiment, the three-dimensional tooth model may be divided into two types, the first type is a closed model, and the second type is a non-closed model. The closed model is formed by stretching an edge and then adding a plane to serve as a bottom surface.

For the non-closed three-dimensional tooth model, the minimum oriented bounding box may be used to surround the three-dimensional tooth model. Two surfaces having a maximum area of the minimum oriented bounding box are the side which the teeth of the three-dimensional tooth model face and the side on which the gum part is away from the teeth. In two normal vectors of the two surfaces having the maximum area of the minimum oriented bounding box, the direction of one normal vector may be selected as the target orientation.

As an optional example, determining the initial orientation of the three-dimensional tooth model according to the minimum oriented bounding box includes: confirming a preset axis direction according to two surfaces having the maximum area of the minimum oriented bounding box; acquiring a first average coordinate value of boundary edges of the three-dimensional tooth model and a second average coordinate value of vertexes of the polygonal patches of the three-dimensional tooth model based on the preset axis direction; and determining the initial orientation according to the first average coordinate value and the second average coordinate value.

Optionally, in the present embodiment, the preset axis may be a preset direction, for example, a coordinate axis or another direction of a three-dimensional space. If any direction is used as the preset direction, the direction of the preset axis is the same as the preset direction.

After the preset axis is determined, the first average coordinate value of the boundary edges of the three-dimensional tooth model and the second average coordinate value of all vertexes may be acquired. The boundary edges are boundaries between the teeth and the gums, the first average coordinate value of the boundary edges may be understood as an average value of segmentation points of the teeth and the gums, and the second average coordinate value may be understood as the center of gravity of the three-dimensional tooth model. The center of gravity of the three-dimensional tooth model deviates towards one side of the tooth regions. Therefore, in the two surfaces having he maximum area of the minimum oriented bounding box that surrounds the three-dimensional tooth model, the surface closer to the second average coordinate value is the side where the teeth are located. Therefore, the initial orientation of the three-dimensional tooth model can be determined.

As an optional example, determining the initial orientation of the three-dimensional tooth model includes: in a case where the three-dimensional tooth model is a closed model, determining a polygonal patch group to which the polygonal patches on the surface of the three-dimensional tooth model belong, wherein a planar included angle between any two polygonal patches in the same polygonal patch group is less than an included angle threshold; and determining the initial orientation of the three-dimensional tooth model according to the sum of the areas of the polygonal patches in the polygonal patch group.

In the present embodiment, the closed model may be obtained by filling the non-closed model, and the closed three-dimensional tooth model may also be obtained by directly performing oral cavity scanning when an oral cavity scanning operation is performed on the interior of the oral cavity. The initial orientation of the closed three-dimensional tooth model may be determined by calculating the area of the polygonal patch group consisting of polygonal patches.

As an optional example, in a case where the three-dimensional tooth model is the closed model, determining the polygonal patch group to which the polygonal patches on the surface of the three-dimensional tooth model belong includes: selecting any first patch from all patches that are not divided into polygonal patch groups to serve as a patch in one polygonal patch group, and determining a polygonal patch of which a planar normal included angle with the first patch is less than the included angle threshold as a patch that is located in the same polygonal patch group with the first patch, wherein in an initial case, all polygonal patches on the three-dimensional dental model are not divided into the polygonal patch groups; and continuing to select any one target patch from all patches that are not divided into the polygonal patch groups to serve as a patch in another polygonal patch group, and determining a polygonal patch of which the planar included angle with the target patch is less than the included angle threshold as a patch that is located in the same polygonal patch group of the target patch, until all polygonal patches are divided into the polygonal patch groups.

As an optional example, in a case where the three-dimensional tooth model is the closed model, determining the polygonal patch group to which the polygonal patches on the surface of the three-dimensional tooth model belong includes: selecting any first patch from all patches that are not divided into polygonal patch groups to serve as a patch in one polygonal patch group, and determining a polygonal patch of which the included angle between the normal thereof and the normal of the first patch is less than the included angle threshold as a patch that is located in the same polygonal patch group of the first patch, wherein in an initial case, all polygonal patches on the three-dimensional dental model are not divided into the polygonal patch groups; and selecting any target patch from all patches that are not divided into the polygonal patch groups to serve as a patch in another polygonal patch group, and determining a polygonal patch of which the included angle between the normal thereof and the normal of the target patch is less than the included angle threshold as a patch that is located in the same polygonal patch group of the target patch, until all polygonal patches are divided into the polygonal patch groups.

In the present embodiment, the polygonal patches may be grouped to obtain different polygonal patch groups, and the planar included angle between the polygonal patches in each polygonal patch group is less than the included angle threshold.

When the polygonal patch group is divided, any polygonal patch among all polygonal patches may be used as one polygonal patch in one polygonal patch group; and then, the polygonal patch is taken as a basic patch, each polygonal patch among all polygonal patches, of which the planar included angle with the polygonal patch is less than the included angle threshold or of which the included angle between the normal thereof and the normal of the polygonal patch is less than the included angle threshold, is added into the polygonal patch group. In the remaining polygonal patches, the planar included angle of each polygonal patch and the planar included angle of the basic patch are greater than or equal to the included angle threshold. The operation is repeated until all polygonal patches belong to one polygonal patch group. The polygonal patches in each one polygonal patch group may be regarded as being located on the same plane. The sum of areas of the polygonal patches in the polygonal patch group is calculated, and the polygonal patches having the maximum sum of areas in the polygonal patch group are regarded as a gum side of the closed model, and the other side is a tooth side, and thus the initial orientation of the three-dimensional tooth model is determined.

As an optional example, extracting the peak points of the gum line includes: determining a coordinate value of each point on the gum line based on the target orientation; and determining the peak points from the gum line according to the coordinate value, wherein the coordinate value of the peak point is greater than the coordinate value of an adjacent point of the peak point on the gum line.

In the present embodiment, since the orientation of the three-dimensional tooth model is adjusted to the target orientation, for a point on the gum line, if the coordinate value on the target direction is greater than that of the adjacent point, the point is used as the peak point.

If the distance between two adjacent peak points is too small, a point with a greater coordinate value may be used as a peak point, and the other point is not used as a peak point, and since the peak point is a silt region between two teeth, the distance between two peak points cannot be too small, and if so, it indicates that the determination of the peak points is wrong.

As an optional example, pairing the peak points to obtain the peak point combination includes: dividing the peak points into a first peak point group and a second peak point group according to a position relationship between the peak points and the three-dimensional tooth model; and using each peak point in the first peak point group as a first peak point, and using the first peak point and a second peak point in the second peak point group as a group of peak point combinations, wherein the second peak point is a peak point in the second peak point group that is closest to the first peak point, and an angle of a connecting line of the first peak point and the second peak point with a dental center line of the three-dimensional tooth model is greater than an angle threshold.

Optionally, in the present embodiment, when the peak points are paired, two peak points, which are closest to each other and the connecting line of which passes through the dental center line, are paired as a pair of peak point combinations. The dental center line is a connecting line of midpoints of the teeth on the three-dimensional tooth model. The peak points passing through the connecting line indicate that the peak points on the two sides of the teeth are paired rather than pairing the peak points on one side of the teeth.

As an optional example, after using each peak point in the first peak point group as the first peak point, and using the first peak point and the second peak point in the second peak point group as the group of peak point combinations, the method further includes: deleting the successfully paired peak points from the first peak point group and the second peak point group; and using each peak point in the second peak point group as a third peak point, and using the third peak point and a fourth peak point in the first peak point group as a group of peak point combinations, wherein the fourth peak point is a peak point in the first peak point group that is closest to the third peak point, and an angle of a connecting line of the third peak point and the fourth peak point with the dental center line of the three-dimensional tooth model is greater than the angle threshold.

In the present embodiment, pairing may be performed one by one starting from the peak points on one side of the teeth of the three-dimensional tooth model, for each peak point on one side of the teeth of the three-dimensional tooth model, a peak point having a minimum distance is selected from the other side of the teeth, and an included angle between the connecting line of two peak points and the dental center line meets a preset threshold condition, the two peak points are combined into a peak point combination, after the pairing is completed, all successfully paired peak points are deleted, and then pairing is performed again starting from the other side of the teeth, so that the pairing of all peak points is realized.

Further, if the peak point having the minimum distance is only searched for according to the distance, a pairing error may occur easily in the case of an oblique tooth or an incisor, and the pairing method may avoid the pairing error in the scenario by using a manner of determining whether the included angle meets the preset threshold condition.

There may be a plurality of pairing failure cases, for example, the distance between two peak points is too large, or one peak point on one side and two peak points on the other side may be paired, etc.

As an optional example, determining the segmentation path between the teeth in the tooth regions based on the peak point combination includes: determining each peak point combination as the current combination; and determining the shortest distance of the two peak points in the current combination on the three-dimensional tooth model as the segmentation path.

After the peak point combination is determined, the segmentation path may be determined according to the peak point combination. The segmentation path may be the shortest path between two peak points on the teeth.

As an optional example, determining the segmentation path between the teeth in the tooth regions based on the peak point combination includes: determining each peak point combination as the current combination; determining the shortest distance of the two peak points in the current combination on the three-dimensional tooth model as a first path; correcting the first path in a manner of overlaying curvatures to obtain a target path; and determining the target path as the segmentation path.

In the present embodiment, the segmentation path may be used as the path for segmenting the teeth, or the segmentation path may also be adjusted, and after the shortest path between the two peak points on the teeth is obtained, the path may also be corrected in the manner of overlaying curvatures, and the corrected path is used as the segmentation path.

As an optional example, correcting the first path in the manner of overlaying curvatures to obtain the target path includes: using each point on the first path as the current point, and determining replacement points of the current point within a preset range on the three-dimensional tooth model, wherein the replacement points are points meeting a curvature threshold requirement; and using the connecting line of the replacement points as the target path.

In the present embodiment, the shortest path is corrected in the manner of overlaying curvatures, that is, the points on the path are locally adjusted according to the curvature, and each point on the shortest path is corrected. Each point is used as the current point, and if there are points meeting the curvature threshold requirement within a preset range in the vicinity of the current point on the three-dimensional tooth model, the points meeting the curvature threshold requirement are used to replace the current point. The replaced points are connected to serve as the segmentation path.

The above three-dimensional tooth model segmentation method is described below in combination with one example.

The three-dimensional tooth model segmentation method of the present disclosure may be mainly applied to a correction presentation scenario, after tooth segmentation, tooth positions are adjusted according to a correction solution, and corrected teeth are generated and displayed.

Further, the tooth segmentation technology may also be applied to the design of the orthodontic guide plate, the design of the substitutive tooth model and the design of the temporary teeth.

In the present embodiment, if the teeth of the user are to be checked or corrected, the oral cavity of the user may be scanned at first to obtain the data of the three-dimensional tooth model of the teeth of the user. The data may be displayed to the user and the doctor by the display screen, so that the doctor can introduce the situations of the teeth for the user.

The three-dimensional tooth model of the user obtained by oral cavity scanning may be a closed or non-closed model. The three-dimensional tooth model includes tooth regions and gum regions, and the boundaries between the tooth regions and the gum regions are not labeled. Thus, if tooth segmentation needs to be performed, a segmentation line (the gum line) between the teeth and the gums and the segmentation path of the teeth need to be determined.

That is, in the present solution, scanning data output by a 3D scanner is acquired; the model is automatically straightened based on identified model features; a feature value of the three-dimensional dental model is extracted based on the automatically straightened model and by means of a geometric algorithm of curvatures, and filtering and denoising are performed on the extracted feature value to obtain a gum line and peak points thereof, and the tooth parts and the gum parts are segmented to obtain pure tooth parts; and pairing is performed by using the peak points of the gum line, and a segmentation path between one tooth and another tooth is determined by the curvature and shortest path method to obtain all segmented teeth.

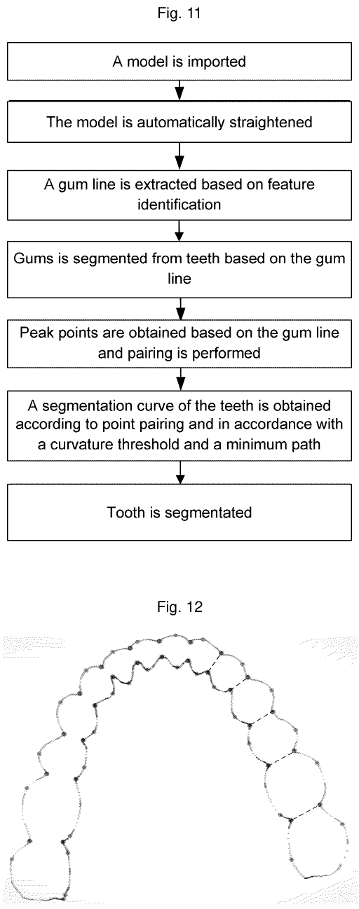

FIG. 11 is an optional flowchart of the present embodiment, and the segmentation of the three-dimensional tooth model is mainly divided into the following steps in the present embodiment:

1. Import the model

Importing the model refers to importing the three-dimensional tooth model. The three-dimensional tooth model is a model obtained by oral cavity scanning. The three-dimensional tooth model is imported into a system, and the system may display the structure of the three-dimensional tooth model via the display screen.

The three-dimensional tooth model may be a scanning model (a non-closed model) output by the 3D scanner, and the input three-dimensional tooth model may be in any direction. In the present solution, any other type of tooth model with a plane does not affect the implementation of the present disclosure.

The three-dimensional tooth model is a digitized three-dimensional body composed of a series of polygonal patches.

2. Automatically straighten the model

After being imported into the model, the three-dimensional tooth model may have different orientations. Therefore, the direction of the three-dimensional tooth model may be first adjusted to the target orientation, so as to straighten the three-dimensional tooth model.

For the imported three-dimensional tooth model, the features thereof are identified, and the purpose of identifying the features is to find the straightening angle of the three-dimensional tooth model, and since different tooth models have different application types, the features thereof are inconsistent, and meanwhile according to different tooth model types, the tooth models may be classified by the extracted the features. During straightening, the initial orientation of the three-dimensional tooth model needs to be determined, and then the initial orientation is adjusted to the target orientation.

There are a plurality of methods for determining the initial orientation. For example, 1). If the model is an oral cavity scanning model, such a model is a non-closed model, a bounding box of a bounding model first be determined by using the Oriented Bounding Box (OBB) technology, and the direction of one normal of two surfaces having the maximum area among six surfaces of the bounding box is determined as a final tooth orientation. This method is to determine the size and direction of the box according to the geometrical shape of an object itself, and the box does not need to be perpendicular to a coordinate axis. In this way, the most suitable and most compact bounding box can be selected.

After the bounding box is obtained, the first average coordinate value of the boundary edges of the three-dimensional tooth model and the second average coordinate value of the vertexes of the polygonal patches of the three-dimensional tooth model are acquired based on the preset axis direction. Then, the side where a plane closer to the second average coordinate value is determined as the side which the teeth face, and the initial orientation of the three-dimensional tooth model is determined.

According to the angle of a target, the bounding box is rotated by using a rotation matrix, so that the model is rotated to the target orientation.

2). If the model is a closed model with a flat bottom surface, straightening is performed in such a manner that a maximum flat bottom surface of the model is close to the bottom, and the method for detecting the maximum plane of a tooth includes: setting a certain polygonal patch, overlaying the set polygonal patch with a 3D tooth model composed of polygonal patches, setting an error threshold e, and when e is greater than a certain value, considering that the set polygonal patch is not flush with the patches on the 3D tooth model; and otherwise, considering that the polygonal patch is located in the same plane. When the set polygonal patch and a certain patch of the tooth model are in the same plane, the polygonal patches are overlaid together, the search for the next polygonal patch is continued, and the error threshold is judged. The above steps are cycled until the maximum plane of the tooth model is obtained. The current normal vector of the maximum plane of the tooth model is determined, and a target normal vector of the maximum plane of the tooth model is acquired.

According to a multiplication cross operation method, a rotation angle and a rotation axis are solved according to vector values before and after rotation (the current normal vector and the target normal vector are solved by using the multiplication cross operation method to obtain the rotation angle and the rotation axis), the multiplication cross operation method is a binary operation of the vectors in a vector space, and an operation result thereof is a vector rather than a scalar; and any model may be rotated to a desired spatial position by the rotation angle and the rotation axis thereof.

3. Extract the gum line based on feature identification

The purpose of identifying and extracting the gum line of the three-dimensional tooth model is to perform tooth segmentation on the three-dimensional tooth model according to the gum line. The gum line may be determined in a variety of manners. For example, the gum line may be determined by a neural network model, the three-dimensional tooth model may be input into the neural network model, and the gum line of the three-dimensional tooth model is labeled by the neural network model. Or, the three-dimensional tooth model may be projected onto a two-dimensional plane, then tooth regions on the two-dimensional plane are identified and correspond to the three-dimensional tooth model, so as to label the boundary between the teeth and the gums, that is, the gum line.

It is taken as an example that the gum line is identified by the neural network model, after the model is placed on a specified position, a feature value of the tooth model is calculated according to the geometric calculation method of curvatures, and filtering and denoising are performed to obtain the contour of the three-dimensional tooth model; and the curvature obtained by the geometric calculation is a rotation rate of the tangential direction angle of the certain surface on the tooth model with respect to the arc length, the curvature indicates the degree of concavity and convexity of the certain surface, and is also referred to as a feature in the present solution. The feature value of a concave-convex region of the tooth model may be acquired by a curvature method (a real gum line on the tooth model is also embodied by concavity and convexity); and noise needs to be removed from an obtained initial feature value of the tooth model, so as to ensure that there are concavity and convexity on other positions of each tooth in addition to the gum line region, which are also regarded as a feature, this part affects the subsequent fitting with a contour line, and thus it needs to be filtered or removed to finally obtain an optimal feature value.

The feature value is simplified into a contour line, that is, an initial gum line, since the initial gum line is not a smooth curve, there will be cases of local folding and deviation, and the initial gum line is processed by using a principal component analysis method to obtain points in a main direction. Whether the points are consistent in the main direction may be determined by using data, for example, where peaks and valleys are located are positions where the curve changes the bending direction, and these positions may be configured to determine the points in the main direction. In this case, in the present solution, the main contour shape is acquired by using the principal component analysis method to determine the point in the main direction of the contour, thereby avoiding an uneven line segment region to obtain a final required smooth gum line.

4. Segment the teeth and the gums based on the gum line

After the gum line is identified, the tooth parts and the gum parts of the three-dimensional tooth model may be segmented according to the position of the gum line. The purpose of segmenting the tooth parts and the gum parts is to make tooth segmentation be more accurate.

5. Obtain peak points based on the gum line and perform pairing

The determination of the points in the main direction of the contour is to determine peak points and valley points. In the present solution, an insertion control point is determined according to a curvature bending degree of the gum line (the peak points and the valley points are all regions that are bent), and since the valley points are always lower than the peak points, the peak points may be judged according to an upper-lower distance (a height difference in the target direction), and the valley points are filtered to obtain complete peak points.

After the peak points are obtained, the two peak points, which have the shortest distance and the connecting line of which passes through the dental center line, are paired to serve as a starting point and an ending point of tooth segmentation. For example, as shown in FIG. 12, in FIG. 12, the peak points on both sides of the teeth are paired. (In FIG. 12, one peak point combination is connected by one dotted line, and not all dotted lines are shown).

6. Obtain a segmentation curve of the teeth according to point pairing and in accordance with a curvature threshold and a minimum path

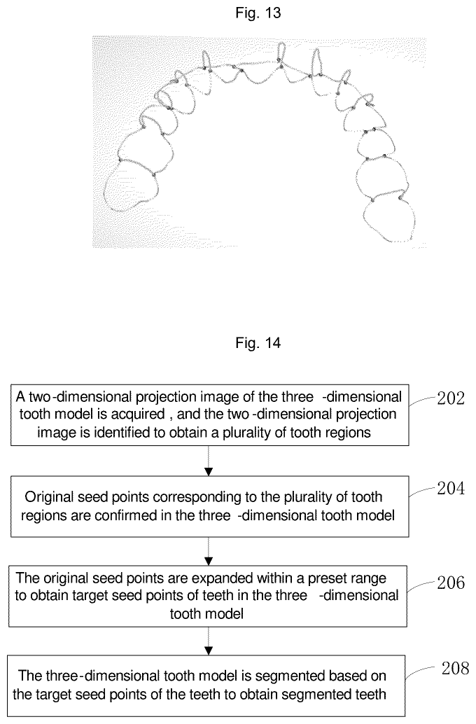

Since one tooth and another tooth are in contact and connected with each other previously, the teeth are cut by the curve to form independent single teeth. In the present solution, an initial cutting curve is first determined in a shortest distance mode, the shown shortest distance is to walk along the surface of a triangular patch of the tooth model starting from a starting peak point that has been paired and reach the ending peak point within the shortest distance. Since the shortest distance is not the best cutting line, the shortest distance is corrected in the manner of overlaying curvatures in the present solution, and since the contact between one tooth and another tooth is uneven, shortest distance segmentation lines are overlaid by two methods within the range of a large curvature to determine a final segmentation line. The determined segmentation path is shown in FIG. 13.

7. Segment the teeth according to the segmentation curve

All teeth are segmented according to the segmentation curve to obtain segmented teeth.

In some other application scenarios, a tooth segmentation operation may be directly performed without depending on the extracted gum line. For example, as shown in FIG. 14, the three-dimensional tooth model segmentation method may include the following steps:

Step S202, a two-dimensional projection image of the three-dimensional tooth model is acquired, and the two-dimensional projection image is identified to obtain a plurality of tooth regions;

Step S204, original seed points corresponding to the plurality of tooth regions are confirmed in the three-dimensional tooth model;

Step S206, the original seed points are expanded within a preset range to obtain target seed points of teeth in the three-dimensional tooth model; and

Step S208, the three-dimensional tooth model is segmented based on the target seed points of the teeth to obtain segmented teeth.

Similarly, the three-dimensional tooth model herein may be understood as the foregoing three-dimensional tooth model. The purpose of the three-dimensional tooth model segmentation method is to accurately segment each tooth on the three-dimensional tooth model into a single tooth.

The two-dimensional projection image may be an image obtained by projecting the three-dimensional tooth model onto a plane. The two-dimensional projection image includes tooth regions and non-tooth regions. By identifying the two-dimensional projection image, the plurality of tooth regions may be identified, so as to perform tooth segmentation on the teeth on the three-dimensional tooth model. The identification method may refer to identifying the two-dimensional projection image in a machine vision or artificial intelligence mode to obtain the tooth regions.

The surface of the three-dimensional tooth model is composed of polygonal patches, such as triangular patches, one triangular patch includes three vertices, and the vertices of all triangular patches may be regarded as seed points. The original seed points are seed points corresponding to the tooth regions in the two-dimensional projection image among all seed points of the three-dimensional tooth model. That is, by means of the original seed points, the tooth regions may be preliminarily determined on the three-dimensional tooth model.

Since the tooth regions determined by the original seed points may be inaccurate, the original seed points may be expanded by a method for expanding the original seed points to obtain the target seed points. Regions covered by the target seed points may be regarded as teeth on the three-dimensional tooth model, and accurate tooth segmentation may be performed on the three-dimensional tooth model via the target seed points.

After the tooth regions are identified, it can be understood that the positions and the regions where the teeth are located have been determined on the two-dimensional projection image. Then, the teeth correspond to the three-dimensional tooth model to determine the original seed points on the three-dimensional tooth model. Since the seed points are the vertexes of the triangular patch on the surface of the three-dimensional tooth model, the original seed points may be understood as regions covering the teeth on the three-dimensional tooth model. In order to ensure the accuracy, the original seed points are further expanded to obtain the target seed points, and the three-dimensional tooth model is segmented according to the target seed points to obtain the segmented teeth, and so far, the teeth in the three-dimensional tooth model may be divided into single teeth.

In the above method, the original seed points on the three-dimensional tooth model are determined by identifying the two-dimensional projection image of the three-dimensional tooth model, and the original seed points are expanded to obtain the target seed points, so that the range of the teeth on the three-dimensional tooth model are marked by the target seed points, and the three-dimensional tooth model may be further segmented according to the target seed points to obtain the segmented teeth, thereby achieving the effect of performing accurate tooth segmentation on the three-dimensional tooth model.

Optionally, in the present embodiment, after the original seed points are obtained, the original seed points may be expanded to obtain the target seed points. The process of expanding the original seed points may be divided into one or more stages.

For example, in one stage, the original seed points may be expanded according to a preset curvature threshold to obtain the target seed points. The preset curvature threshold may be understood as a constraint condition used when the original seed points are expanded, thereby avoiding the expansion of the original seed points exceeding a limit. The preset curvature threshold may include one or more curvature values, and if the preset curvature threshold includes one curvature value, the original seed points may be expanded according to the curvature value to obtain the target seed points, and if the preset curvature threshold includes a plurality of curvature values, the first curvature value may be used to expand the original seed points, then the second curvature value is used to expand an expansion result of the first curvature value, and the third curvature value is used to expand the expansion result of the second curvature value, until all curvature values are used once.

As an optional example, expanding the original seed points according to the preset curvature threshold to obtain the target seed points includes: expanding the original seed points according to an initial curvature threshold to obtain first seed points; and expanding the first seed points according to a target curvature threshold to obtain the target seed points, wherein the target curvature threshold is obtained according to the initial curvature threshold.

It is taken as an example that the preset curvature threshold is composed of the initial curvature threshold and the target curvature threshold, the original seed points are first expanded by using the initial curvature threshold to obtain the first seed points, and then the first seed points are expanded by using the target curvature threshold to obtain the target seed points. The initial curvature threshold and the target curvature threshold may be the same or different, and the target seed points are obtained by twice successive expansions.

As an optional example, expanding the original seed points according to the initial curvature threshold to obtain the first seed points includes: using seed points adjacent to the original seed points as current seed points; and in a case where the curvatures of the current seed points are less than or equal to the initial curvature threshold, using the current seed points and the original seed points as the first seed points.

In the present embodiment, when the original seed points are expanded by using the initial curvature threshold, the curvatures of the seed points adjacent to the original seed points may be acquired. The curvature is a rotation rate of a tangential direction angle of a certain point on a curve with respect to an arc length, which is defined by differentiation, indicates the degree of the curve deviating from a straight line and indicates a numerical value of the degree of bending of the curve at a certain point. Each seed point corresponds to one curvature. By comparing magnitude relationships between the curvatures and the initial curvature threshold, it is determined whether the seed points adjacent to the original seed points may be taken as the first seed points. The original seed points may be used as the first seed points without comparing the curvatures.

Adjacency points mentioned herein and below may be understood as two vertexes that form the same edge of the same triangular patch together with a seed point. For example, adjacency points to the original seed point refer to two vertices that form the same edge of the same triangular patch together with the original seed point.

As an optional example, expanding the first seed points according to the target curvature threshold to obtain the target seed points includes: using seed points adjacent to the first seed points as current seed points; and in a case where the curvatures of the current seed points are less than or equal to the target curvature threshold, using the first seed points and the current seed points as the target seed points.

After the original seed points are expanded by using the initial curvature threshold to obtain the first seed points, the first seed points may be expanded by using the target curvature threshold to obtain the target seed points. The curvatures of the seed points adjacent to the first seed points are determined, then the curvatures are compared with the target curvature threshold, and by means of comparing the magnitude relationship, it is determined whether the seed points adjacent to the first seed points are the target seed points. So far, the original seed points are expanded by using the initial curvature threshold and the target curvature threshold to obtain the target seed points.

As an optional example, before expanding the first seed points according to the target curvature threshold to obtain the target seed points, the method further includes: using the sum of the initial curvature threshold and a preset value as the target curvature threshold, wherein the preset value is a positive number.

Optionally, in the present embodiment, the initial curvature threshold and the target curvature threshold may be empirical values, or the initial curvature threshold is an empirical value, and the target curvature threshold is determined according to the initial curvature threshold. For example, the sum of the initial curvature threshold and the preset value is used as the target curvature threshold, that is, the target curvature threshold is obtained according to the initial curvature threshold, and the target curvature threshold is greater than the initial curvature threshold. The preset value is a preset value, and may be modified according to different three-dimensional tooth models.

As an optional example, after expanding the original seed points according to the initial curvature threshold to obtain the first seed points, or expanding the first seed points according to the target curvature threshold to obtain the target seed points, the method further includes: adjusting the first seed points as non-first seed points in a case where corresponding points of the first seed points on the two-dimensional projection image do not fall into corresponding tooth regions; or, adjusting the target seed points as non-target seed points in a case where corresponding points of the target seed points on the two-dimensional projection image do not fall into the corresponding tooth regions.

Optionally, in the present embodiment, when the original seed points are expanded according to the initial curvature threshold or the first seed points are expanded according to the target curvature threshold, it is also necessary to check whether the expanded seed points meet requirements, that is, whether the expansion exceeds a range, whether seed points in non-tooth regions are used as the first seed points or whether the seed points in the non-tooth regions are used as the target seed points. For example, when the original seed points are expanded according to the initial curvature threshold, if the curvatures of the seed points adjacent to the original seed points are less than or equal to the initial curvature threshold, it is also necessary to judge regions where the corresponding points of the seed points on the two-dimensional projection image are located. If the corresponding points of the seed points are not located in the tooth regions, it indicates that the seed points have deviated from the tooth regions of the three-dimensional tooth model, and thus the seed points are used as the non-first seed points. When the first seed points are expanded according to the target curvature threshold, if the curvatures of the seed points adjacent to the first seed points are less than or equal to the target curvature threshold, it is also necessary to judge regions where the corresponding points of the seed points on the two-dimensional projection image are located. If the corresponding points of the seed points are not located in the tooth regions, it indicates that the seed points have deviated from the tooth regions of the three-dimensional tooth model, and thus the seed points are used as the non-target seed points.

As an optional example, after expanding the original seed points according to the curvature threshold to obtain the target seed points, and before segmenting the three-dimensional tooth model based on the target seed points of the teeth to obtain the segmented teeth, the method further includes: expanding the plurality of tooth regions to obtain target regions; and expanding the target seed points in the target regions according to the initial curvature threshold and heights.

After the original seed points are expanded to obtain the target seed points, the teeth of the three-dimensional tooth model may be segmented according to the target seed points. In addition, before the teeth are segmented, the target seed points may be expanded again. In other words, in addition to expanding the original seed points in the first stage by using the initial curvature threshold and the target curvature threshold, to obtain the target seed points, the expansion in the second stage may also be performed.

During the expansion in the second stage, the tooth regions on the two-dimensional projection image may be adjusted at first, and the tooth regions are enlarged to obtain the target regions. Then, with the target regions as a limitation, the target seed points are expanded in the second stage by using the initial curvature threshold and the heights. The purpose of expanding the tooth regions to the target regions is to ensure that the seed points located on the teeth on the three-dimensional tooth model are marked as the target seed points, thereby avoiding omission.

As an optional example, when the target seed points are expanded according to the initial curvature threshold and the heights, the seed points adjacent to the target seed points may be used as the current seed points; and when the heights of the current seed points are greater than a preset standard height and the curvatures of the current seed points are less than or equal to the target curvature threshold, the current seed points are used as the target seed points.

The height may be a numerical value of the seed point in a preset direction of the three-dimensional tooth model. For example, the preset direction of the three-dimensional tooth model may be used as the Z axis, and a coordinate value of the seed point on the Z axis is used as the height of the seed point.

The preset direction may be any direction. After the three-dimensional tooth model is obtained, the orientation of the three-dimensional tooth model may be adjusted to the preset direction, so that the directions of all acquired three-dimensional tooth models can be unified.

When the target seed points are expanded by using the initial curvature threshold and the heights, the seed points adjacent to the target seed points may be used as the current seed points, and if both the curvatures and the height values of the current seed points meet the requirements of the initial curvature threshold and the heights, the current seed points may be used as the target seed points, thereby completing the expansion of the target seed points.

As an optional example, in a case where the heights of the current seed points are greater than the preset standard height and the curvatures of the current seed points are less than or equal to the target curvature threshold, using the current seed points as the target seed point includes: when the heights of the current seed points are greater than the preset standard height and the curvatures of the current seed points are less than or equal to the target curvature threshold, and when the corresponding points of the current seed points on the two-dimensional projection image are located in the target regions, using the current seed points as the target seed points, wherein the target regions are regions obtained by expanding the plurality of tooth regions; and in a case where the corresponding points of the current seed points on the two-dimensional projection image are located outside the target regions, using the current seed points as non-target seed points.

Optionally, when the target seed points are expanded by using the initial curvature threshold and the heights, it should be ensured that the expanded target seed points do not exceed the target regions. The target regions are regions obtained after the tooth regions are expanded. The purpose of expanding the tooth regions is to encompass a part of non-tooth regions in the vicinity of the tooth regions in the target regions, so that when the target seed points are expanded by using the initial curvature threshold and the heights, the target seed points can be allowed to be expanded to the non-tooth regions in the vicinity the tooth regions. In this way, the expanded target seed points may cover all tooth regions.

As an optional example, after expanding the target seed points according to the initial curvature threshold and the heights, the method further includes: using seed points adjacent to the expanded target seed points as current seed points; and using the current seed points as the target seed points as well.

After the target seed points are expanded by using the initial curvature threshold and the heights, the expanded target seed points may be expanded once again, and the seed points adjacent to the target seed points are also used as the target seed points. The purpose of this expansion is also to enable the expanded target seed points to cover all tooth regions, so that when the teeth are segmented according to the expanded target seed points, the teeth are complete.

As an optional example, before expanding the original seed points within the preset range, the method further includes: expanding the plurality of tooth regions to obtain the target regions; marking points other than points on the three-dimensional tooth model of the target regions as third seed points; and expanding the third seed points according to the curvature threshold.

In the present embodiment, the tooth regions are firstly expanded to obtain the target regions, so that the target regions include all tooth regions and also include the non-tooth regions in the vicinity of the tooth regions. Therefore, when the points other than the points on the three-dimensional tooth model of the target regions are marked as the third seed points, the third seed points are not points on the teeth. By expanding to obtain the third seed points, points of non-tooth parts (gaps between gum parts or the teeth) that are close to tooth parts may be marked as the third seed points, so that the boundaries between parts beyond the teeth and the tooth parts may be close to the tooth parts, and the range of the target regions is reduced, therefore although the target regions include the non-tooth regions, the included non-tooth regions are fewer.

After the third seed points are expanded, the segmentation lines between the tooth parts and the parts beyond the teeth on the three-dimensional tooth model become “thinner”.

As an optional example, expanding the third seed points according to the curvature threshold includes: using seed points adjacent to the third seed points as current seed points; and when the curvatures of the current seed points are greater than the curvature threshold, using the current seed points as the third seed points.

In the present embodiment, when the third seed points are expanded, the third seed points may be expanded according to the curvatures and the curvature threshold. The curvature of the third seed point may be obtained by calculating by differentiation by means of calculating the rotation rate of the tangential direction angle of the point with respect to the arc length. The curvature threshold may be a preset threshold, and for different three-dimensional tooth models, the curvature threshold may be different.

As an optional example, after using the seed points adjacent to the third seed points as the current seed points, the method further includes: in a case where corresponding points of the current seed points in the two-dimensional projection image are located in the target regions, using the current seed points as non-third seed points.

After the third seed points are expanded, it is also necessary to check whether the third seed points are expanded into the target regions. Since the target regions include the tooth regions, if the third seed points are expanded into the target regions, the third seed points may be expanded into the tooth regions, so the third seed points located in the target regions are returned as the non-third seed points.

As an optional example, after expanding the third seed points according to the curvature threshold, the method further includes: determining a first region formed by the third seed points; determining a sub-region from the first region; and in a case where the sub-region is surrounded by the target seed points, determining the seed points in the sub-region as the target seed points.

Optionally, in the present embodiment, after the third seed points are expanded, the third seed points constitute the first region. The first region may be divided into a plurality of sub-regions. Each sub-region among the plurality of sub-regions may be understood as a region composed of a plurality of third seed points. If a certain sub-region is surrounded by the target seed points, it indicates that the sub-region is located within the tooth parts on the three-dimensional tooth model, but the sub-region may not be a tooth, that is, a hole part on the tooth. Therefore, the seed points in the sub-region are used as the target seed points and are divided as the tooth parts.

As an optional example, acquiring the two-dimensional projection image of the three-dimensional tooth model includes: adjusting the orientation of the three-dimensional tooth model from the initial orientation to the target orientation; and projecting the three-dimensional tooth model in the target orientation onto a target plane to obtain the two-dimensional projection image.

Optionally, in the present embodiment, when the three-dimensional tooth model is projected into the two-dimensional projection image, the orientation of the three-dimensional tooth model may be adjusted first to adjust the orientation to the target orientation. The target orientation may be a preset orientation. The purpose of adjusting the orientation of the three-dimensional tooth model is that, when the three-dimensional tooth model is projected into the two-dimensional projection image, the tooth regions on the two-dimensional projection image may be more complete and less shielded. For example, a horizontal plane may be used as the X axis and the Y axis of three-dimensional coordinates, an upward direction of the horizontal plane is used as the target orientation, and after the three-dimensional tooth model is acquired, the tooth direction of the three-dimensional tooth model may face the target orientation.

After the orientation of the three-dimensional tooth model is adjusted, the three-dimensional tooth model may be projected to the target plane, and the target plane may be the horizontal plane.

As an optional example, identifying the two-dimensional projection image to obtain the plurality of tooth regions includes: inputting the two-dimensional projection image into an identification model, and marking the plurality of tooth regions on the two-dimensional projection image by the identification model.

Optionally, in the present embodiment, the identification model may identify the two-dimensional projection image, so as to identify the tooth regions. After the two-dimensional projection image is input into the identification model, the identification model extracts and identifies features, and outputs the plurality of tooth regions.

As an optional example, confirming, in the three-dimensional tooth model, the original seed points corresponding to the plurality of tooth regions includes: using the vertex of each triangular patch in the three-dimensional tooth model as the current vertex; and in a case where a corresponding point of the current vertex in the two-dimensional projection image is located in the plurality of tooth regions, using the current vertex as one original seed point.

Optionally, in the present embodiment, the surface of the three-dimensional tooth model is covered by polygonal patches, the polygonal patches may be triangular patches, and the triangular patch is taken as an example, and each triangular patch has three vertices. Two adjacent triangular patches share one edge. In the vertexes of each triangular patch, points corresponding to the two-dimensional projection image are located in the tooth regions, and then the vertexes may be used as the original seed points.

As an optional example, after segmenting the three-dimensional tooth model based on the target seed points of the teeth to obtain the segmented teeth, the method further includes: arranging the segmented teeth.

As an optional example, arranging the segmented teeth includes: using an average value of tooth midpoints of all teeth as a starting point, and using the tooth midpoint of each tooth as an ending point to form a vector of each tooth; using, as a target straight line, a straight line where two teeth in all teeth having a maximum distance between the tooth midpoints are located; and arranging all teeth according to the size of an included angle between the vector and the target straight line.

Optionally, in the present embodiment, the teeth may be arranged after being segmented. Taking connecting lines between the tooth midpoints and the midpoint of each tooth serve as vectors, teeth may be arranged according to the included angle between the vectors and the target straight line formed by the two teeth having the maximum distance in all teeth. That is, tooth arrangement is starting from the first tooth on a certain side of the teeth to the last tooth on the other side.

As an optional example, the manufacturing method of the dental device includes at least one of the following steps: performing tooth segmentation processing on the three-dimensional tooth model, or performing arrangement processing on the teeth to obtain a tooth model to be formed.

As an optional example, after segmenting the three-dimensional tooth model based on the target seed points of the teeth to obtain the segmented teeth, the method further includes: smoothing the edges of the teeth to obtain smoothed edges.

As an optional example, smoothing the edges of the teeth to obtain the smoothed edges includes: sorting vertexes on the edges of the teeth; using a second vertex in the sorted vertices as the current vertex, and performing the following operations on the current vertex until the current vertex does not include a back vertex: using a center point of a front vertex and a center point of the back vertex of the current vertex as smoothing points; every time when a smoothing point is obtained, using the back vertex of the current vertex as a new current vertex, and using the obtained smooth point as the front vertex of the new current vertex; and connecting the first vertexes on the edges of the teeth with the obtained smoothing points in sequence to obtain the smoothed edges of the teeth.

In the present embodiment, the purpose of smoothing the edges of the teeth is to make corner angles of segmentation line parts of the teeth and parts outside the teeth be smaller, thereby avoiding the situation in which a subsequently cut tooth model is too sharp to cause discomfort in the gums of the user.

During the smoothing, among the seed points through which the edges of the teeth pass, a series of seed points form a curve. Starting from a starting seed point, an average value with a second seed point is calculated to obtain a first midpoint, an average value between the first midpoint and a third seed point is calculated to obtain a second midpoint, and an average value between the second midpoint and a fourth seed point is calculated to obtain a third midpoint. The above step is repeated until the last seed point on the edges of the teeth is processed. All midpoints are connected in sequence to obtain the smoothed edges of the teeth.

The above three-dimensional tooth model segmentation method is described below in combination with one example.

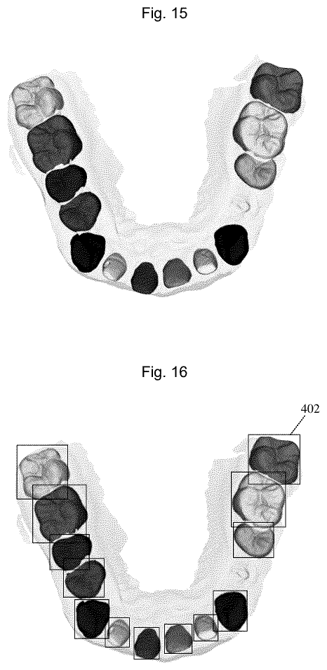

For example, as shown in FIG. 15, FIG. 15 illustrates a two-dimensional projection image of an exemplary three-dimensional tooth model. The teeth and gum parts in FIG. 15 form the three-dimensional tooth model obtained by oral cavity scanning, and the three-dimensional tooth model is projected onto a plane to obtain the two-dimensional projection image as shown in FIG. 15.

After the oral cavity scanning data of the user is acquired to obtain the three-dimensional tooth model of the user, the direction of the three-dimensional tooth model may be adjusted. The purpose of adjusting the three-dimensional tooth model is that when the two-dimensional projection image is obtained by projection, the tooth parts of the three-dimensional tooth model may appear on the two-dimensional projection image as many as possible.

When the direction is adjusted, the three-dimensional tooth model may be input, and the three-dimensional tooth model is straightened in a positive direction of the Z axis. The straightening method may utilize any of the straightening methods in the art, which is not excessively limited herein. It is taken an example that the teeth of the three-dimensional tooth model are placed on a table top upwards, then the table top may be the X axis and the Y axis of a three-dimensional rectangular coordinate system, and a normal on the side of the table top facing the teeth is the Z axis. After the three-dimensional tooth model is straightened in the direction, the teeth of the three-dimensional tooth model face upwards, and FIG. 15 may be a top view of the three-dimensional tooth model, that is, a two-dimensional projection image obtained by projecting the three-dimensional tooth model onto the table top.

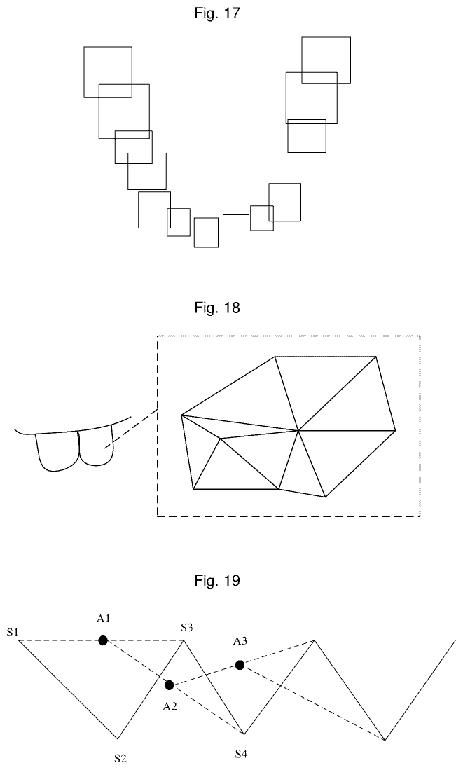

After the three-dimensional tooth model is projected onto the plane to obtain the two-dimensional projection image, the two-dimensional projection image may be identified by using the identification model, so that the tooth regions on the two-dimensional projection image can be identified. The identified tooth regions may be marked with boxes. Each box may be a minimum axial bounding box including a mask region, or may be an inclined box. The identified tooth region may have a regular shape or an irregular shape, for example, a block 402 in FIG. 16 is the identified tooth region. The tooth regions include tooth regions and non-tooth regions. The blocks in FIG. 16 are merely examples. For example, as shown in FIG. 17, FIG. 17 illustrates the identified tooth regions.

After the tooth regions are identified from the two-dimensional projection image, actual tooth regions in the three-dimensional model may be determined. Corresponding to the three-dimensional tooth model, the tooth regions on the two-dimensional projection image may correspond to the tooth parts on the three-dimensional tooth model. All points on the tooth parts are filtered to remove points having an intersection with the original model in the normal direction, and remaining points after filtering are used as original seed points.

For example, as shown in FIG. 18, the surface (the tooth parts and the non-tooth parts, and the surface of the entire model) of one three-dimensional tooth model (only two incisors are shown in FIG. 18) is formed by combining triangular patches (which may also be quadrilateral patches, pentagonal patches . . . or other polygonal patches). The triangular patches are not completely located on the same plane, and there are planar included angles between each other. The vertexes of the triangular patches are seed points. Or, after noise is removed from the vertexes of the triangular patches, the remaining points serve as seed points. If corresponding points of the seed points on the three-dimensional tooth model on the two-dimensional projection image are located in the tooth regions as shown in FIG. 16, the seed points are used as the original seed points.

In the present embodiment, the original seed points may be expanded to obtain target seed points. The purpose of extending the original seed points is that the original seed points may not include all tooth parts on the three-dimensional tooth model, therefore the tooth parts of the three-dimensional tooth model are covered by expansion.

The expansion of the original seed points are divided into a plurality of stages.

In a first stage, the original seed points are expanded by curvatures. In practice, the expansion of the original seed points is to check whether there are seed points capable of serving as target seed points together with the original seed points in seed points adjacent to the original seed points.

Whether the seed points are adjacent to each other may be judged by judging whether the seed points are located on the same straight line.

When the original seed points are expanded by the curvatures, two successive expansions may be performed. The expansion may be first performed by using an initial curvature threshold, and then the expansion is performed by using a target curvature threshold. The target curvature threshold is the sum of the initial curvature threshold and a preset value. Therefore, for seed points adjacent to the original seed points, it is first judged whether the curvatures are less than the initial curvature threshold, and if the curvatures are less than the initial curvature threshold, then the seed points and the original seed points are used as first seed points together. Then, for the seed points adjacent to the first seed points, if the curvatures are less than or equal to the target curvature threshold, and then the seed points and the first seed points are used as the target seed points together. Therefore, the two successive expansions of the original seed points are completed. At this time, the expansion in the first stage has not ended. Because of the expansion in the first stage, the original seed points have been expanded twice successively, therefore there may be too many expanded points and thus exceeding the tooth parts. At this time, it is necessary to judge whether corresponding positions of the expanded points on the two-dimensional projection image fall into the tooth regions. If the corresponding positions of the expanded points on the two-dimensional projection image fall into the tooth regions, it indicates that the expansion of the original seed points on the three-dimensional tooth model does not exceed the tooth parts. If the corresponding positions of the expanded points on the two-dimensional projection image do not fall into the tooth regions, the corresponding seed points are no longer used as the target seed points. So far, the expansion in the first stage is ended.

In a second stage, the target seed points in the first stage may be expanded by using the initial curvature threshold and heights. In the second stage, seed points adjacent to the target seed points obtained after the expansion in the first stage may be used as seed points to be expanded, the curvatures of this part of seed points is less than or equal to the initial curvature threshold, and the heights thereof are greater than a preset standard height. If the two conditions are both met, this part of seed points also is used as the target seed points. If one condition is not met, this part of seed points is not used as the target seed points. In one specific example, the preset standard height may be the height of the current point.

Moreover, in addition to the curvatures and the heights of this part of seed points meeting the conditions, this part of seed points should also be located in target regions on the two-dimensional projection image. The target regions are expanded regions of the tooth regions. That is, on the two-dimensional projection image, the tooth regions are slightly expanded to obtain the target regions. Then, during the expansion in the second stage, if the expanded seed points exceed the target regions on the two-dimensional projection image, the seed points exceeding the target regions are not used as the target seed points, that is, the expansion is rolled back. So far, the expansion in the second stage is completed.

That is to say, the seed points of each tooth are firstly expanded according to a given curvature threshold, and it is ensured that the seed points do not exceed the range of the tooth regions on the two-dimensional projection image, and these points are used as initial points of each tooth; and the expansion is performed again on the initial points according to a target curvature threshold (by adding 0.2 to the given curvature threshold), and it is ensured that the expansion does not exceed the range of the bounding box of the tooth regions.

The expansion is performed according to the given curvature threshold and the heights, and it is ensured that the expansion does not exceed the range of the bounding box of the tooth regions+an expanded range (i.e., the target regions); and the boundary point of each tooth extends outwards once (extending to the vicinity of a silt and a gum line), and an extension distance may be preset.

The initial seed points are not all regions of final teeth and thus need to be continuously diffused. In the present solution, a diffusion range is formed according to two-dimensional projection. The regions (the bounding box of the tooth regions+the expanded range) refer to a range to which the seed points is able to be diffused, and the regions (the bounding box of the tooth regions+the expanded range) form the diffusion range according to the two-dimensional projection image, and the diffusion range may be controlled by algorithm parameters.

The reason for expanding the tooth regions for multiple times is that the partition between adjacent teeth in some tooth models is not obvious. When the tooth regions are expanded, the height is defined at last to reduce the possibility of the teeth expanding downwards to the gums. The reason for expanding the boundaries of the teeth outwards once is that after segmentation is performed directly according to the curvature threshold, the teeth obtained by segmentation are smaller than the original sizes by one circle, which is caused by a small curvature in the vicinity of the gum line, and thus the extension is required.

In addition to the expansion in the first stage and the second stage, parts outside the teeth may also be expanded. That is, on the three-dimensional tooth model, points outside the target regions on the two-dimensional projection image are used as third seed points, wherein the third seed points are points outside the tooth parts, for example, points of the gum parts or silts between the teeth. This part of seed points is to be expanded towards the tooth parts. At the time of expansion, the expansion may be performed according to curvatures. If the curvatures of seed points adjacent to the third seed points are greater than a required curvature threshold, this part of seed points and the third seed points are used as the third seed points together, that is, the points outside the tooth parts. However, it should be noted that if the expanded third seed points are located in the target regions on the two-dimensional projection image, the seed points may be expanded to the tooth parts, therefore rollback is required, and the third seed points located in the target regions are used as non-third seed points.

With regard to the target seed points obtained by the expansion in the first stage and the second stage, and the third seed points obtained after the expansion, the boundaries thereof may be used as the boundaries of the teeth.

If the teeth include pores, there may be a bounding case between the target seed points and the third seed points, for example, the target seed points surround a part of the third seed points, this is because there are pores in the teeth, and the pores are identified as the third seed points. In this case, the points in the regions surrounded by the target seed points are also used as the target seed points.

After the expansion is completed, the teeth are segmented according to the target seed points and the third seed points, to obtain single teeth. Alternatively, before the teeth are segmented, the boundaries of the teeth are smoothed at first, and the smoothed boundaries are smoother. For example, as shown in FIGS. 19, S1 to S4 are points on the boundaries of the teeth, a midpoint between S1 and S3 is A1, a midpoint between A1 and S4 is A2, . . . , and all midpoints A1 to An are connected to obtain the smoothed boundaries.

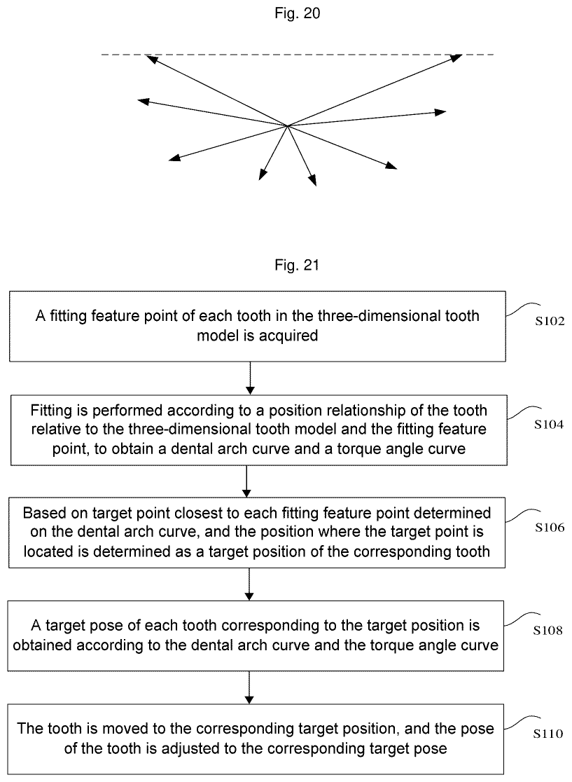

After the teeth are segmented to obtain the single teeth, the teeth may be arranged. Taking the three-dimensional tooth model as an example, the arrangement is shown in FIG. 20. An average value of tooth midpoints of the three-dimensional tooth model is used as a starting point, the tooth midpoint of each tooth is used as an ending point, and a plurality of vectors may be obtained by connection. A straight line connecting two teeth having a maximum tooth midpoint is used as a target straight line (a dotted line in FIG. 20), included angles formed by the vectors and the target straight line are checked, and arrangement is performed according to the sizes of the included angles.

In addition, in some embodiments, the pre-processing operation may further include slicing the digitized tooth model, adding a bottom plate, hollowing out the bottom plate, adding a tooth model fixing accessory, adding identification information, and the like, but it is not limited to all of the foregoing steps (i.e., gum line extraction, tooth segmentation, tooth arrangement (i.e., the tooth arrangement mentioned above), slicing, adding the bottom plate, hollowing out the bottom plate, adding the tooth model fixing accessory, adding the identification information, and the like), and at least one may be selected therefrom according to actual needs.

Step 3, performing forming processing based on the pre-processed digitized tooth model to obtain a solid dental model.

In one embodiment, 3D printing may be performed based on the digitized tooth model or a tooth model obtained after the foregoing processing operation, to obtain the solid dental model, wherein the 3D printing may be photocuring 3D printing, such as SLA, DLP, LCD or other 3D printing modes, such as 3DP, MJF, FDM and Polyjet.

In some embodiments, the solid dental model obtained based on the foregoing pre-processing operation may have a hollowed-out bottom plate, a fixing accessory, identification information, and the like.

Optionally, after 3D printing, a post-processing step is further included, wherein the post-processing step may be selected according to the utilized 3D printing mode. For example, when the photocuring 3D printing technology is utilized, the post-processing step may be post-curing, cleaning or other steps.

Of course, in other embodiments, the forming mode is not limited to 3D printing, and other forming modes, such as injection molding, may also be utilized, which is not limited herein.

Step 4, performing film pressing processing on the solid dental model.

After the solid tooth model is obtained, the film pressing processing may be performed on a preheated polymer material film on the solid tooth model, to obtain a shell-shaped film covering the solid tooth model, that is, an initial equipment that has not been cut. For example, the shell-shaped film at least covers the tooth parts of the solid tooth model.

In one embodiment, an image of the solid tooth model may be collected by using an image collection terminal, such as a CCD image sensor, so as to identify the identification information via OCR and other identification technologies, then a corresponding film pressing instruction is called from a database based on the identification information, and the film pressing processing is performed based on the film pressing instruction, wherein the film pressing instruction may include a pressure parameter, a diaphragm preheating time, a film pressing temperature, and the like.

Step 5, performing a marking operation on the shell-shaped diaphragm.

In the step, the marking operation is mainly performed on the shell-shaped film based on the identification information that is added in the pre-processing operation, so as to form an identifier on the shell-shaped film to obtain an initial equipment with the identifier.

For example, similar to the foregoing steps, an image of the solid tooth model may be collected by using an image collection terminal, such as a CCD image sensor, then the identification information is identified via OCR and other identification technologies, and then a corresponding marking instruction is called based on the identification information to mark the shell-shaped film based on parameter information contained in the marking instruction.

It should be noted that, in the manufacturing method of the dental device, the execution of the step is not limited, that is, whether the marking operation is needed may be selected according to actual needs.

Step 6, cutting the initial equipment to obtain the dental device.

In the step, the initial equipment is mainly cut based on a cutting line obtained by converting the extracted gum line, so that useless parts in the initial equipment are cut off and only useful parts are reserved, to obtain the dental device. For example, the dental device may be an invisible appliance.

It should be noted that in the step, the acquisition of the cutting line may be the same as that in the foregoing embodiment of the manufacturing method of the dental device, and for related detail content, reference may be made to the foregoing implementations, thus details are not described herein again. In addition, the sequence of step 5 and step 6 is not limited in the present embodiment.

In one embodiment, similar to the foregoing steps, an image of the solid tooth model may be collected by using an image collection terminal, such as a CCD image sensor, then the identification information is identified via OCR and other identification technologies, and then a corresponding cutting instruction is called based on the identification information to cut the initial equipment based on parameter information contained in the cutting instruction.

It should also be noted that in the current application scenario of invisible orthodontic tooth diagnosis and treatment, due to the cross fusion of computer aided design (CAD) and oral medicine, the virtual tooth correction technology becomes a research hotspot. A digital tooth model is arranged by using the computer technology, on one hand, the orthodontic movement direction of each tooth in a three-dimensional space is analyzed to serve as a reference for the doctor to formulate a treatment plan, and on the other hand, the arrangement process of the digitized three-dimensional tooth model is visually presented, so that a patient can know the correction result in advance, therefore it is of great significance to study a tooth arrangement process.

Traditionally, a tooth arrangement work is done manually. MOTOHASHI and KURODA proposed in 1999 to separate a digitized dental jaw according to single teeth and complete tooth arrangement in a manual mode. A common method for manual tooth arrangement is to define a tooth arrangement coordinate system and a local tooth coordinate system, and generate a dental arch curve for a malocclusion dental arch according to clinical experience as assistance, so as to manually pull the teeth in upper and lower jaws to the vicinity of the dental arch curve. If this process is operated by a well-trained tooth arrangement technician, about 15-20 minutes are required, and the tooth arrangement is affected by visual errors.

In summary, although the manual tooth arrangement may meet the requirements of orthodontic treatment, the efficiency is very low.

In view of this, according to a first aspect of the embodiments of the present disclosure, further provided is a tooth arrangement method of a three-dimensional tooth model, and optionally, as shown in FIG. 21, the method includes:

S102, a fitting feature point of each tooth in the three-dimensional tooth model is acquired;

S104, fitting is performed according to a position relationship of the tooth relative to the three-dimensional tooth model and the fitting feature point, to obtain a dental arch curve and a torque angle curve;

S106, a target point closest to each fitting feature point on the dental arch curve is determined, and the position where the target point is located is determined as a target position of the corresponding tooth; S108, a target pose of each tooth corresponding to the target position is obtained according to the dental arch curve and the torque angle curve; and S110, the tooth is moved to the corresponding target position, and the pose of the tooth is adjusted to the corresponding target pose.

The tooth arrangement method of the three-dimensional tooth model may be applied to the process of performing tooth arrangement on a tooth model. The three-dimensional tooth model may be an oral cavity scanning model obtained by performing oral cavity scanning on teeth. For example, before the teeth are corrected, the three-dimensional tooth model is obtained by scanning the teeth, and then a corrected model is obtained by automatic tooth arrangement, so that the teeth are corrected according to the corrected model.

In one specific example, the three-dimensional tooth model may be a stereolithography (STL) model, and the model may include a plurality of triangular patches, or may include other polygonal patches, wherein each triangular patch includes a three-dimensional coordinate of each vertex and a normal vector of the triangular patch. The three-dimensional tooth model may be a three-dimensional model in which the teeth have been segmented, or a three-dimensional model in which the teeth have not been segmented. For example, in a case where the three-dimensional tooth model is a three-dimensional model in which the teeth have been segmented, the three-dimensional tooth model may be directly used for acquiring the fitting feature point of each tooth. In a case where the three-dimensional tooth model is a three-dimensional model in which the teeth have not been segmented, after tooth segmentation is performed on the three-dimensional tooth model by using any tooth segmentation means in the related art, the fitting feature point of each tooth is acquired based on the three-dimensional tooth model that has been subjected to tooth segmentation. For example, the position relationship may be obtained by establishing a coordinate system, for example, a total coordinate system of the three-dimensional tooth model and a local coordinate system of the tooth are established; and then the position relationship is obtained according to the total coordinate system and the local coordinate system.

In one embodiment, the tooth arrangement process of the present disclosure may be divided into the following main stages: 1, importing of the three-dimensional tooth model; 2, straightening of the three-dimensional tooth model; 3, tooth arrangement of the three-dimensional tooth model; and 4, collision detection of the three-dimensional tooth model.

In one embodiment, regarding the process of importing the three-dimensional tooth model, reference may be made to the following description:

The importing of the three-dimensional tooth model may include: importing an STL model of each tooth that has been segmented to serve as the three-dimensional tooth model, or obtaining the three-dimensional tooth model by methods such as oral cavity scanning. The imported three-dimensional tooth model may be a tooth model at any placement position, and the tooth model is a digitized three-dimensional body composed of a series of vertices and triangular patches. These tooth models may be named according to FDI (Federation Dentaire Internationale) in advance.

A coordinate system into which the three-dimensional tooth model is imported may be used as the total coordinate system. The total coordinate system may be a preset coordinate system, and the three-dimensional tooth model is placed in the total coordinate system, so that each point on the three-dimensional tooth model has coordinates in the total coordinate system.

In one embodiment, regarding the process of straightening the three-dimensional tooth model, reference may be made to the following description:

In the present embodiment, after the three-dimensional tooth model is placed in the total coordinate system, the position and orientation of the three-dimensional tooth model may be adjusted first according to the total coordinate system, and the purpose of adjusting the position and orientation is to “straighten” the three-dimensional tooth model, so as to provide a basis for subsequent tooth arrangement. The straightening may be to automatically straighten to the origin of the coordinate system, and an occlusal surface of the three-dimensional tooth model is aligned with an x-y plane. When the three-dimensional tooth model is “straightened”, a center point and vectors (a first vector and a second vector) of the three-dimensional tooth model may be determined, and during the straightening process, the center point of the three-dimensional tooth model may be adjusted to the origin of the total coordinate system, the first vector coincides with the Z axis of the total coordinate system of the three-dimensional tooth model, and the second vector coincides with the X axis of the total coordinate system, so that the “straightening” of the three-dimensional tooth model is completed.

The center point, the first vector and the second vector need to be calculated.

The acquisition mode of the center point may be as follows: traversing the teeth of the three-dimensional tooth model in a sequence from an incisor to a last molar of the three-dimensional tooth model, and determining the centroid of each tooth on the three-dimensional tooth model; and using, as the center point, the center of a first centroid connecting line of the centroid of the traversed first tooth having no missing teeth on left and right sides and the centroid of the traversed last tooth having no missing teeth on the left and right sides.

First, the center point of a bounding box of each tooth is calculated. The centroid of the tooth may be determined via the center point of the bounding box (the center point may be regarded as the centroid, or the center point is offset according to the volume of the tooth in each region of the bounding box, to obtain the centroid). When the center point (an FP point) is calculated, an iterative search may be performed in a sequence from the first incisor to the last molar, if there are no missing teeth on identical tooth positions on the left and right sides of a tooth, the tooth is used as a useful tooth, and the center of the centroid connecting line of the first useful tooth and the last useful tooth is used as the center point.

The vector may be acquired as follows: traversing the teeth of the three-dimensional tooth model in a sequence from the last molar to the incisor of the three-dimensional tooth model, and determining the centroid of each tooth on the three-dimensional tooth model; determining, as the second vector, a second centroid connecting line of the centroid of the traversed first tooth having no missing teeth on the left and right sides and the centroid of the traversed last tooth having no missing teeth the on left and right sides; determining the center of the second centroid connecting line as an auxiliary point; determining a vector from the auxiliary point to the center point as an auxiliary vector; and determining a product of the second vector and the auxiliary vector as the first vector. In other words, teeth meeting a condition may be iteratively searched in the sequence from the first incisor to the last molar, the condition is that there are no missing teeth on identical tooth positions on the left and right sides, and then a midpoint of the centroid connecting line of first two teeth meeting the above requirement is used as the center point (the FP point). Afterwards, teeth meeting a condition may be iteratively searched in the sequence from the last molar to the first incisor, the condition is that there are no missing teeth on identical tooth positions on the left and right sides, and then the midpoint of the centroid connecting line of first two teeth meeting the above requirement is used as the auxiliary point (an EP point), and the centroid connecting line is used as the second vector [symbol] .

For example, the second vector is expressed as [symbol] , the first vector is expressed as [symbol] , the auxiliary point is expressed as EP, and the auxiliary vector is expressed as [symbol] .

The EP point and the [symbol] vector are calculated as follows: an iterative search is performed from the last molar to the first incisor, if there is one tooth having no missing teeth on identical tooth positions on the left and right sides, the midpoint of the centroid connecting line of first two teeth meeting the above requirement is used as the auxiliary point (the EP point), and the centroid connecting line is used as the second vector [symbol] . [symbol] and [symbol] are calculated as follows: the vector from FP to EP is [symbol] , and [symbol] = [symbol] × [symbol] is calculated by multiplication cross.

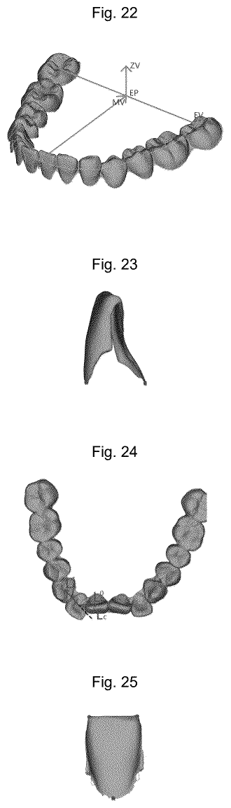

After the center point, the first vector and the second vector are calculated, [symbol] is transformed to the positive direction of the Z axis, and meanwhile [symbol] is transformed to a rotation target in the positive direction of the X axis, a rotation matrix is calculated, rotation transformation is performed. A translation matrix from a transformed point FP′ to the origin is calculated by performing rotation transformation on FP, and the translation matrix is applied to a whole-jaw model. In this way, the center point coincides with the total coordinate system, the first vector is consistent with the positive direction of the Z axis, and the second vector is consistent with the positive direction of the X axis. FIG. 22 is a schematic diagram of parameters during straightening.

In one embodiment, regarding the tooth arrangement process of the three-dimensional tooth model, reference may be made to the following description:

Prior to tooth arrangement, smoothing filtering may be performed on each tooth. Different 3D model smoothing methods may be used, and a method in a computational geometry algorithms library (CGAL) is used in the present embodiment. Of course, other filtering algorithms may also be used, as long as the smoothing filtering may be implemented, which is not limited in the present disclosure.

During tooth arrangement, it is necessary to determine a target point of each tooth on the dental arch curve and a target pose at the target point.

For each tooth on the three-dimensional tooth model, a fitting feature point may be determined from the vertexes of the triangular patch of the tooth, and then the dental arch curve is fitted according to the fitting feature point of each tooth. A pre-established ideal dental arch curve model may be solved according to the fitting feature point to obtain a correction parameter, wherein the correction parameter includes a lateral translation amount, a longitudinal translation amount and a rotation angle; the position of the ideal dental arch curve model is straightened according to the correction parameter, to obtain an ideal dental arch model; and curve fitting is performed according to the ideal dental arch model, to obtain the dental arch curve. After the dental arch curve is determined, a target point closest to the fitting feature point is determined on the dental arch curve. The target point is a point corresponding to the fitting feature point on the dental arch curve. It should be noted that in a specific implementation, there is case where an initial position of the tooth has an overlapping part with the dental arch curve, but the fitting feature point is not on the dental arch curve. Since the fitting feature point is on the tooth, the fitting feature point is moved while the tooth is moved, therefore the process of moving the tooth to the target position is a process of moving the fitting feature point to the target point. It can be understood that the tooth is moved in a mode from the fitting feature point to the target point, so as to move tooth to the target position.

Although the tooth is moved to the target position, the pose and the orientation of the tooth have not been adjusted. Therefore, it is necessary to determine the target pose of the tooth. The target pose is affected by a variety of factors. The target pose of the tooth may be determined according to the total coordinate system of the three-dimensional tooth model, the local coordinate system of the tooth, the dental arch curve and the torque angle curve. During the process of determining the target pose of the tooth, the torque angle curve is introduced, and during the process of determining the torque angle curve, if there is a missing tooth on the three-dimensional tooth model, the fitting feature point of the tooth on a tooth-missing position may be simulated by the data of a tooth on the symmetrical tooth position on the saw jaw, so as to determine the torque angle curve, therefore the target pose can be generated relatively accurately even if there is the missing tooth on the three-dimensional tooth model, and the tooth is adjusted to the target pose to complete accurate automatic tooth arrangement. Compared with the existing automatic tooth arrangement methods, the present disclosure compensates for the tooth data of the tooth-missing part via the simulation data and then performs curve fitting, thereby being able to adapt to the tooth-missing case, and thus improving the accuracy of tooth arrangement.

The local coordinate system may be determined by the normal vectors of the vertexes of the triangular patches on the teeth.

The dental arch curve may be fitted by the feature points of each tooth on the three-dimensional tooth model. For the feature points of each tooth, each tooth may be used as the current tooth; a first feature point and a second feature point of the current tooth are determined; according to the type to which the current tooth belongs, a third feature point is determined for the current tooth by using a method matching the type; and the feature point of the current tooth is determined from the first feature point, the second feature point and the third feature point.

Determining the first feature point and the second feature point may include: intercepting a target vertex and a polygonal patch from the three-dimensional tooth model after the three-dimensional tooth model is straightened; determining a linear vector of the target vertex; determining a target bounding box of the target vertex and the polygonal patch, wherein an X-axis direction of the target bounding box is the direction of the linear vector; dividing the three-dimensional tooth model into a cheek-side part and a tongue-side part according to the target bounding box; calculating a first bounding box of the three-dimensional tooth model of the cheek-side part; and using, as the first feature point and the second feature point of the current tooth, two points closest to two endpoints on the top of the first bounding box in the vertexes of the three-dimensional tooth model of the cheek-side part.

Determining the third feature point may include: in a case where the current tooth is a first-type tooth, using, as the third feature point of the current tooth, a point closest to the bottom of the first bounding box in the vertexes of the current tooth in the three-dimensional tooth model of the cheek-side part; in a case where the current tooth is a second-type tooth, using, as the third feature point of the current tooth, a point closest to the top of the first bounding box in the vertexes of the current tooth in the three-dimensional tooth model of the cheek-side part; in a case where the current tooth is a third-type tooth or a fourth-type tooth, extracting a second bounding box of the current tooth; determining a first vertex set and a second vertex set from the second bounding box; determining a pit and fissure direction of the current tooth according to the second vertex set; adjusting the direction of the second bounding box to be consistent with the pit and fissure direction; dividing the current tooth into four parts according to the adjusted second bounding box; in a case where each part of the current tooth includes the vertexes in the first vertex set, using a vertex in the vertexes in the first vertex set that is closest to the top of the second bounding box as the third feature point of the current tooth; and in a case where each part of the current tooth does not include the vertexes in the first vertex set, traversing the vertexes from one vertex in the current part of the current tooth to the center of the second bounding box, and using a point in the traversed vertexes that is closest to the top of the second bounding box as the third feature point of the current tooth. In the present embodiment, the first type, the second type, the third type and the fourth type are types obtained by classifying teeth according to the positions of the teeth. For example, the first type is an incisor, the second type is a cuspid tooth, the third type is a premolar, and the fourth type is a posterior molar.

When the first feature point, the second feature point and the third feature point are determined, a tooth direction guide vector may be extracted at first to obtain a proximal direction vector of a tooth in the horizontal direction, so as to guide a place where the proximal direction vector is required, for example, the oriented bounding box (OBB). The main steps are as follows:

For each tooth, bottom sharp points Pp (as shown in FIG. 23) of a cheek side and a tongue side are extracted, and the specific process is as follows: the contour of the boundary of the tooth is extracted, and the contour is defined as that only one side of an edge thereon has a triangle. The contour is smoothed to overcome the impact caused by a plurality of sawteeth on the boundary during tooth segmentation. An optimal oriented bounding box (OBB) is extracted for the smoothed contour. Points that are closest to the bottom of the bounding box and are located on two contours on diagonal phases are extracted, that is, P_{b}.

For each tooth, a center connecting line vector L_{c} is extracted (as shown in FIG. 24). L_{c}⁰ is calculated at first, which is a vector between the tooth and the center of the bounding box of an adjacent tooth in a proximal direction, then L_{c}¹ is calculated, which is a vector between the tooth and the center of the bounding box of an adjacent tooth in a distal direction, and if the adjacent tooth is missing, the operation is performed the next adjacent tooth in sequence until the vector is found. Then, L_{c} is calculated by using the following formula:

$${L}_{c} = 0.5 * ( {L}_{c}^{0} - {L}_{c}^{1} )$$

For each tooth, which one of the two points P_{b} is the cheek side and which one is the tongue side are deduced via an included angle of a vector between the two points P_{b} with L_{c}.

After the cheek side and the tongue side are determined, the incisors, the cuspid teeth, the premolars and the posterior molars among the teeth may be determined, so as to calculate feature points according to different teeth.

For the feature points of the incisors and the cuspid teeth, a top region may be intercepted: vertexes and patches (generally 20%) of a region of a certain proportion on the top are extracted according to the height of the Z axis, a linear direction [symbol] of these vertexes is calculated by using a 3D linear fitting algorithm, and then a bounding box B composed of these vertexes and patches is extracted by using the direction [symbol] as the X-axis direction of the bounding box. The tooth model is cut along a middle plane of the bounding box B on the top, so as to divide the tooth model into two parts, that is, the cheek side and the tongue part. The model of the cheek-side part is extracted and a bounding box thereof is calculated, and two points in the vertexes thereof that are closest to the two endpoints of the top of the bounding box are used as the first feature point (a distal end) and the second feature point (a proximal end).

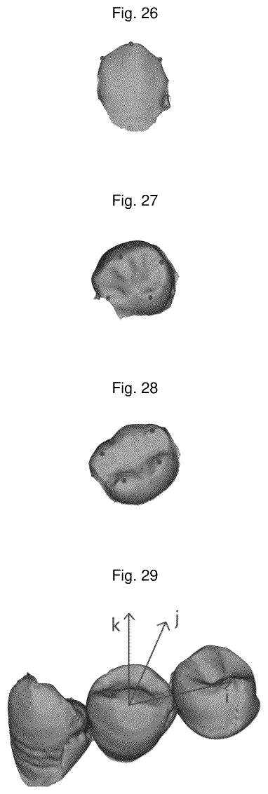

If the tooth is an incisor, a point in the vertexes of the cheek-side model that is closest to the bottom of the bounding box is used as the third feature point, and if the tooth is a cuspid tooth, a point in the vertexes that is closest to the top of the bounding box is used as the third feature point. FIG. 25 and FIG. 26 respectively illustrate three feature points of the incisor and three feature points of the cuspid tooth.

The first feature points and the second feature points of the premolar and the molar are consistent with those of the incisor and the cuspid tooth, and the third feature point may be extracted as follows:
1). Extracting a bounding box of a top region of the premolar, wherein the specific steps thereof are as follows:
   A. performing curvature calculation on the molar to obtain a curvature value and the direction of each vertex: PV₁ (maximum curvature value), PV₂ (minimum curvature value), [symbol] (maximum curvature direction), [symbol] (minimum curvature direction).
   B. extracting a mesh part in which PV₁ is greater than a threshold Thₜₒₚ, and the Z value is higher than a half-height of the bounding box, and considering the mesh part as the top region of the tooth; and further judging the number of the vertexes of the obtained top region, if the number of the vertexes is less than 10, it indicates that the tooth itself is too short or too small, and then considering the entire tooth as the top region.
   C. in order to avoid the interference of non-tooth parts (for example, tissues around the toot are also scanned) in the surrounding, extracting a maximum communication region for one time.
   D. performing further filtering processing on the extracted region:
      a. extracting an optimal oriented bounding box (OBB).
      b. calculating a normal direction of each vertex, calculating an included angle with a normal vector of a top surface of the OBB, and filtering the vertex if the included angle exceeds a threshold (usually 60 degrees).
      c. performing filtering again according to the height to filter a lower half part of the OBB and only reserve an upper half part.
2). Extracting a convex point set and a concave point set from the top region, wherein the convex point set may be used for subsequently extracting the feature points, and the concave point set may be used for extracting the pit and fissure direction (which is an alternative method and is not finally used in the present disclosure). The definition of the convex point set is:

$$\{ V \vert V \vert {PV}_{1} > {Th}_{top} and {PV}_{2} > {Th}_{convex} \}$$

The definition of the concave point set is:

$$\{ V \vert {PV}_{1} > {Th}_{top} and {PV}_{2} < {Th}_{concave} \}$$

3). Extracting the pit and fissure direction, wherein the specific steps thereof are as follows:
   A. updating the OBB according to the guidance of L_{c}, so that the x axis of the local coordinate system of the OBB is consistent with L_{c}.
   B. establishing a uv coordinate system for the top of the OBB, wherein a u axis is the x axis of the local coordinate system of the OBB, and a v axis is the y axis of the local coordinate system of the OBB. Sampling is performed in two directions, then 3D straight line fitting is performed on obtained points, and the result of which direction is better is judged by a fitting result, and then the better fitting straight line is the pit and fissure direction of the molar (meanwhile, it is agreed that the pit and fissure direction points from the distal end to the proximal end). The sampling process in the two directions is as follows:
      a. performing sampling along the u axis at a certain interval, and finding a point with the lowest model surface height along the v axis at each u value.
      b. performing sampling along the v axis at a certain interval, and finding a point with the lowest model surface height along the u axis at each v value.
4). Guiding the update of the direction of the OBB by using the pit and fissure direction, so that the x axis of the local coordinate system of the OBB is consistent with the pit and fissure direction.
5). Calculating the feature points of the premolar/molar:
   A. dividing the top surface of the tooth into four quadrants according to the OBB, wherein the difference between the premolar and the molar lies in the difference between angle intervals for dividing the four quadrants.
   B. in each quadrant, if the convex point set is not empty, finding out a convex point closest to the top surface of the OBB in the quadrant to serve as the feature point of the quadrant; if the convex point set is empty, using one search mode to search for the point closest to the top surface of the OBB from a fixed position of the quadrant to the center of the OBB, so as to serve as the feature point of the quadrant, and if the point has not been found after the search is completed, establishing a feature point by using the fixed position to avoid error reporting of a program; and using the feature point as the third feature point. FIG. 27 and FIG. 28 respectively illustrate the third feature points of the premolar and the posterior molar.

After the feature points of each tooth are determined, the fitting feature point is determined from the feature points, and then the dental arch curve is fitted by the fitting feature point. In the present embodiment, each tooth is used as the current tooth; in a case where the current tooth is the first-type tooth or the fourth-type tooth, a midpoint of a connecting line between the first feature point and the second feature point of the current tooth is used as the fitting feature point; in a case where the current tooth is the second-type tooth, the third feature point of the current tooth is used as the fitting feature point; and in a case where the current tooth is the third-type tooth, the first feature point of the current tooth is used as the fitting feature point. For example, for the incisor, the midpoint of the connecting line between the first feature point and the second feature point is calculated as the fitting feature point. For the cuspid tooth, the third feature point is used as the fitting feature point. For the premolar, the first feature point is used as the fitting feature point. For the molar, the midpoint of the connecting line between the first feature point and the second feature point is used as the fitting feature point.

In one implementation, performing fitting according to the position relationship of the tooth relative to the three-dimensional tooth model and the fitting feature point, to obtain the dental arch curve includes:
fitting and solving a pre-established ideal dental arch curve model according to the fitting feature point, to obtain an initial dental arch curve and a correction parameter, wherein the correction parameter includes a lateral translation amount, a longitudinal translation amount and a rotation angle; and
adjusting the position of the initial dental arch curve according to the correction parameter, to obtain the dental arch curve, wherein the dental arch curve is located on the ideal dental arch curve model.

In one implementation, in a case where there is no target tooth on a tooth position of the three-dimensional tooth model, the fitting feature point of the target tooth may also be obtained according to simulation data of the target tooth, wherein the simulation data is obtained by simulating the target tooth by using data of a tooth on a symmetrical tooth position on the same jaw as the target tooth. In the present disclosure, the tooth data of the tooth-missing part is complemented by the simulation data, and then the dental arch curve is fitted, thereby being able to adapt to the tooth missing case, and thus improving the accuracy of tooth arrangement.

For example, for a dental arch curve fitting method, the pre-established ideal dental arch curve model in the present embodiment is an axisymmetric ideal dental arch curve model, shown in the formula as follows:

$$y = {D ( \frac{2 x}{W} )}^{2 e}$$

wherein D is a dental arch depth; Wis a dental arch width; and a parameter e may be obtained via the position of the cuspid tooth.

Considering that in actual orthodontic treatment, the movement amount of a posterior tooth is much less than that of an anterior tooth, each tooth is weighted to obtain the following expression:

$${min}_{f} {\sum }_{i = 1}^{m} {{ω}_{i} ( {σ}_{i} )}^{2} = {\sum }_{i = 1}^{m} {{ω}_{i} ( f ( {x}_{i} ) - {y}_{i} )}^{2}$$

wherein f(xᵢ) is a selected function model, ω is a weight, the weight assigned to the posterior tooth is higher than that of the anterior tooth, and m is the number of teeth in a single jaw.

In the present disclosure, considering that there may be some errors during the straightening process, the method for fitting the dental arch curve will solve and correct these errors at the same time. Therefore, three unknown correction parameters are added, which are respectively a lateral translation amount xₛ (a translation amount x from the actual dental arch curve to the ideal dental arch curve), a longitudinal translation amount yₛ (a translation amount y from the actual dental arch curve to the ideal dental arch curve), and a rotation angle θ (the rotation angle from the actual dental arch curve to the ideal dental arch curve). By using a nonlinear optimal solving method (e.g., a Ceres Solver), a curve fitting problem is solved by using the fitting feature point, and Xₛ, yₛ,θ, D, W and e may be solved.

Fine tuning is performed on the position of the model, the position of the model is further straightened according to the values of Xₛ, yₛ and θ, so that the dental arch curve is located on an ideal dental arch model only consisting of three parameters D, W and e, so as to obtain a final dental arch curve.

In another implementation, due to medical considerations, it is necessary to widen the dental arch curve prior to tooth arrangement. For example, after performing fitting according to the position relationship of the tooth relative to the three-dimensional tooth model and the fitting feature point, to obtain the dental arch curve, the method further includes:
adjusting y coordinate values of the fitting feature points corresponding to the second-type tooth and the third-type tooth according to a preset proportionality coefficient; and
performing fitting on the dental arch curve again.

Exemplarily, for the obtained dental arch curve, the dental arch curve is widened prior to tooth arrangement, and the main flows are as follows:
adjusting the y coordinates of the fitting feature points of the cuspid tooth and the premolar as: y=0.7*y; and
performing fitting on the dental arch curve again.

It should be noted that the proportionality coefficient is selected according to a specific widening size of the dental arch curve, and the smaller the y coordinate of the fitting feature point is, the wider the dental arch curve is. In the present embodiment, the proportionality coefficient is set as 0.7, and in other embodiments, it may also be set as 0.6, 0.8, or the like, as long as the dental arch curve can be widened.

In the present embodiment, the torque angle curve may be fitted by the total coordinate system, the local coordinate system of the tooth, and the fitting feature point of the tooth. For example, an x value of the fitting feature point of the tooth, and an included angle between a second coordinate axis of the local coordinate system of the tooth and a first coordinate axis of the total coordinate system are determined; and fitting processing is performed on the x value of the fitting feature point and the included angle to obtain the torque angle curve.

If there is a missing tooth on the three-dimensional tooth model, the tooth data of the tooth-missing position may be simulated by using the data of the tooth on the symmetrical tooth position on the same jaw. A first coordinate value of the tooth on the symmetrical tooth position on the same jaw as the target tooth under the total coordinate system is determined; a second coordinate value is determined as the coordinate of the target tooth, wherein the second coordinate value is symmetrical to the first coordinate value with respect to a target plane, and the target plane is a plane in which the coordinate value of the first coordinate axis of the total coordinate system is zero. That is, if there is a missing tooth on the three-dimensional tooth model, the coordinate of the tooth on the tooth position on the same jaw as the tooth-missing position is symmetrically transformed by a symmetrical plane, so as to obtain the coordinate of the tooth on the tooth-missing position.

For example, the current local coordinate system of each tooth may be first calculated and is expressed as [symbol] , [symbol] and [symbol] . In a specific implementation, for the incisor and the cuspid tooth, a vector pointing from the first feature point to the second feature point is used as a [symbol] axis, and a vector from the point P_{b} on the cheek side to the fitting feature point is used as a [symbol] axis.

For the premolar and the posterior molar, a vector in the pit and fissure direction is used as the [symbol] axis, and a local Z-axis vector of the OBB in the top region is used as the [symbol] axis.

A \vec{j} axis is calculated by using

[symbol]

= [symbol] × [symbol] .

The x value of the fitting feature point of each tooth and an included angle γ between the [symbol] axis and the z axis are collected to fit the torque angle curve, and the result is shown in FIG. 29. In order to overcome the influence of the missing tooth, if there is a tooth on the symmetrical tooth position of the same jaw data of the tooth-missing position, a piece of virtual data (symmetrical with respect to a z=0 plane) is made with reference thereto for fitting. In the present disclosure, a symmetric second-order polynomial equation is used for fitting, that is,

γ=ax²+c.

After the total coordinate system of the three-dimensional tooth model, the local coordinate system of the tooth, the dental arch curve and the torque angle curve are obtained, the target pose of the target tooth may be determined. After the target pose is determined, the pose of the tooth may be adjusted according to the target pose. The position of the tooth may be adjusted before or after or at the same time as the adjustment of the pose.

A point P_{c} closest to the Spee curve from the current fitting feature point is calculated.

The pose of the tooth on the point P_{c} is calculated, a [symbol] axis is in the tangential direction on the point P_{c} (at this time, the Spee curve is not fitted yet, therefore it is not necessary to consider the gradient direction of [symbol] along the Spee curve), and the direction of [symbol] is determined and jointly calculated by two conditions, that is, being perpendicular to [symbol] and meeting the torque angle curve with a Z-axis angle.

A transformation matrix required for transforming the tooth from the current pose to the pose on the point P_{c} is calculated and applied. It is also necessary to calculate the pose transformation every time when the position of the tooth is adjusted.

When the pose of the tooth is adjusted, whether the adjustment of the pose is completed may be checked according to a condition. The condition may be that the direction of the tooth on the first coordinate axis of the local coordinate system is consistent with the tangential direction of the dental arch curve at the target point, the direction of the tooth on the second coordinate axis of the local coordinate system is perpendicular to the first coordinate axis, and that an angle between the tooth and the first coordinate axis of the total coordinate system meets the torque angle curve.

So far, the adjustment of the position and pose of each tooth on the three-dimensional tooth model is preliminarily completed.

In one embodiment, regarding the collision detection process of the three-dimensional tooth model, reference may be made to the following description:

After the preliminary adjustment is completed, collision detection is performed to further adjust the position of each tooth. The collision detection is mainly divided into following several steps. 1, adjusting the tooth in the X-axis direction by the collision detection; 2, after the adjustment in the X-axis direction, adjusting the tooth in the Z-axis direction; 3, performing the adjustment in the first step again; 4, adjusting the upper jaw in the Y-axis direction according to the upper jaw and lower jaw occlusion relationship; and 5, adjusting the tooth in the Z-axis direction according to the collision detection, performing the adjustment in the first step again, and performing iteration for multiple times.

The first step may include: horizontally moving the first tooth in each tooth region to a position colliding with the target plane while keeping the pose unchanged, wherein the target plane is a plane in which X=0; using each tooth behind the first tooth in each tooth region as the current tooth, and performing the following operation: in a case where a previous tooth of the current tooth exists, adjusting the pose of the current tooth via the collision detection between the current tooth and the previous tooth, which is specifically as follows:
(1) Preparing a virtual patch of the plane in which x=0 for the collision detection of a tooth #1.
(2) Horizontally moving the tooth #1 in each tooth region to the position just colliding with the plane in which x=0, and keeping the pose of the tooth on a corresponding tooth position on the dental arch curve.
(3) For each tooth region, starting from a tooth 2 #, sequentially checking whether the previous tooth thereof exists, and if so, adjusting the pose of the current tooth on the dental arch curve by performing collision detection with the previous tooth (since the Spee curve is not fitted yet, it is not necessary to consider the gradient direction of [symbol] along the Spee curve).

It is assumed that there are two teeth A and B, and the pose adjustment flow of A on the dental arch curve via the collision detection of A to B is as follows (the same below):
1) Detecting whether A collides with B; if the A does not collide with B, moving A in a direction towards B for 0.1 mm; and if A collides with B, moving A in a direction away from B for a certain distance.
2) Repeating 1) until a detection result is different from an initial detection result.
3) Shortening the moving distance to 0.01 mm, and repeating (1) again until the detection result is different from the initial detection result.

The second step may include: determining the Spee curve according to the coordinate of the fitting feature point of each tooth, wherein in a case where there is a missing tooth, the fitting feature point of the tooth-missing position is simulated by using the fitting feature point of the tooth on the symmetrical position of the tooth-missing position; according to a Z coordinate value of each tooth on the Spee curve, determining whether to adjust the Spee curve; and adjusting the Z-axis direction of the tooth according to the adjusted Spee curve, which is specifically as follows:
(1) Extracting the current coordinate of the fitting feature point of each tooth, and in order to overcome the influence of the missing tooth, if there is a tooth on the symmetrical tooth position of same-jaw data of the tooth-missing position, making a virtual coordinate with reference thereto for fitting.
(2) Solving a Spee curve fitting problem by using the Ceres Solver, wherein a symmetric second-order polynomial equation is used in the present disclosure, that is,

y=a+cx²+ex⁴.
(3) Judging whether the obtained Spee curve is too steep, wherein the judgment standards are as follows:
1) Height differences between Z values of the incisor and the cuspid tooth on the Spee curve and adjacent teeth should be less than 0.5 mm.
2) The difference between the Z value of the premolar on the Spee curve and the value of the Spee curve on x=0 should be less than 2.0 mm.

If it is detected that the Spee curve is too steep, the Spee curve is adjusted in the following method, iterative adjustment and detection are performed until the Spee curve is smooth:

$$\{ \begin{matrix}c = c * 0.8 \\ e = c * 0.8\end{matrix}$$

The third step includes: for each tooth region, starting from the tooth 1 #, sequentially checking whether the previous tooth thereof exists, and if so, adjusting the pose of the current tooth on the dental arch curve by performing collision detection with the previous tooth. For the tooth 1 #, the collision detection with the plane in which x-0 is performed. The difference from step 8 and step 9 lies in that the Spee curve has been fitted at this time, therefore when the pose is adjusted, [symbol] should follow the gradient direction of the Spee curve.

The fourth step may include: adjusting the upper jaw in the Y-axis direction according to the upper jaw and lower jaw occlusion relationship, so that the upper jaw and lower jaw occlusion relationship is appropriate.
(1) According to that the centers of P_{b} of the cheek side and the tongue side of the incisor in the upper jaw have the same (x, y) coordinates as the fitting feature point of a tooth position corresponding to the lower jaw, a parallel movement amount of the upper jaw is calculated. Considering that there may be a missing tooth, the tooth #1 to the tooth #5 are checked in sequence until a result is obtained.

The upper jaw is moved according to a calculation result in (1), so that the upper jaw and the lower jaw have a good occlusion relationship.

The fifth step may include: moving the upper jaw of the three-dimensional tooth model upwards along the Z axis for a predetermined distance, wherein the predetermined distance is a difference value between a fitting feature point with a minimum Z value in the upper jaw and a fitting feature point with a maximum Z value in the lower jaw; and moving each tooth in the upper jaw downwards via the collision detection until colliding with the teeth in the lower jaw, which is specifically as follows:
(1) Calculating the coordinates of fitting feature points of all current teeth, extracting the minimum z value in the upper jaw and the maximum z value in the lower jaw, deducing the upward moving distance of the upper jaw model, and applying same.
(2) During the downward adjustment process, firstly using the teeth in the upper jaw as an entirety, and moving the entity downwards according to a collision detection result until just colliding with the teeth in the lower jaw.
(3) Performing collision detection and movement on the teeth in the upper jaw one by one in the vertical direction.

Every time after the collision in the vertical direction is adjusted, the adjustment in the horizontal direction is performed again, and iteration is performed for several times.

After the collision detection and the adjustment on the positions of the teeth are performed, an adjusted three-dimensional tooth model is obtained. The adjusted three-dimensional tooth model may be used as a tooth arrangement result.

In summary, the present disclosure has the following advantages:
1) The present disclosure provides an automatic straightening method for the three-dimensional tooth model, which may automatically straighten to the origin of the coordinate system, so that the occlusal surface is flush with the x-y plane, thereby facilitating the subsequent extraction of the feature points, and further reducing errors;
2) for the diversity of form changes in the teeth, the automatic extraction method for tooth feature points provided in the present disclosure may identify and extract the feature points of various types of teeth, thereby having high adaptability;
3) the present disclosure provides an axisymmetric curve fitting function, and intelligently uses the data of a tooth-including position to compensate for the data of the tooth-missing position in the case of tooth missing, and then performs solving, thereby improving the accuracy of tooth arrangement;
4) when the dental arch curve is fitted, solving the translation and rotation of the dental arch curve is added, thereby avoiding some errors that may exist in the straightening process and further improving the precision of curve fitting; and
5) the present disclosure provides a robust and efficient orthodontic arrangement method, which may obtain better arrangement and occlusion results, so that the user satisfaction is high.

It should be noted that, regarding the foregoing method embodiments, for simplicity of description, they are expressed as a series of action combinations, but those skilled in the art should know that the present disclosure is not limited by the described action sequence, because some steps may be performed in other orders or simultaneously according to the present disclosure. In addition, those skilled in the art should also know that the embodiments described in the specification are all preferred embodiments, and the involved actions and modules are not necessarily required in the present disclosure.

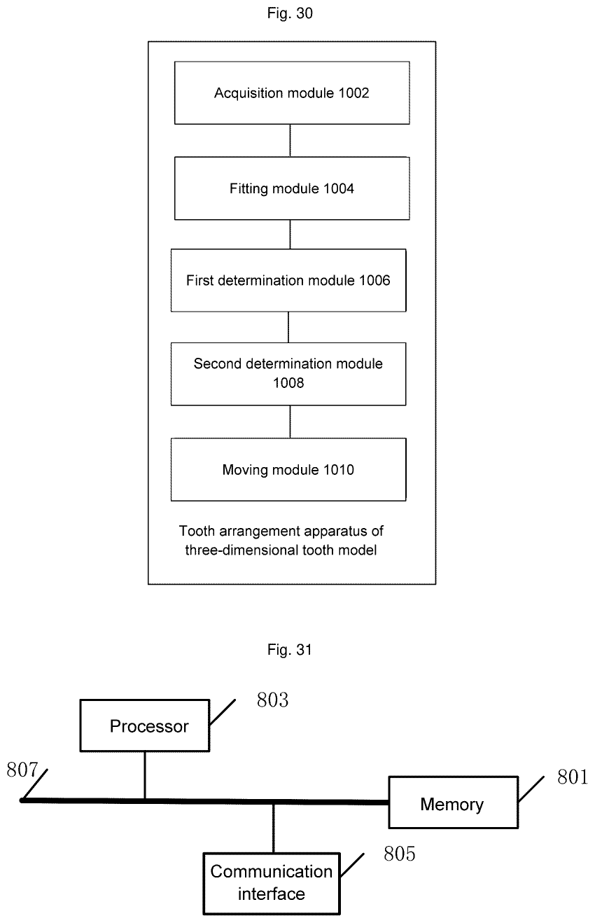

According to another aspect of the embodiments of the present disclosure, further provided is a tooth arrangement apparatus of a three-dimensional tooth model, as shown in FIG. 30, including:

an acquisition module 1002, configured to acquire a fitting feature point of each tooth in the three-dimensional tooth model;

a fitting module 1004, configured to perform fitting according to a position relationship of the tooth relative to the three-dimensional tooth model and the fitting feature point, to obtain a dental arch curve and a torque angle curve;

a first determination module 1006, configured to determine a target point closest to each fitting feature point on the dental arch curve, and determine the position where the target point is located as a target position of the corresponding tooth;

a second determination module 1008, configured to obtain a target pose of each tooth corresponding to the target position according to the dental arch curve and the torque angle curve; and a moving module 1010, configured to move the tooth to the corresponding target position, and adjust the pose of the tooth to the corresponding target pose.

For other examples of the present embodiment, reference may be made to the foregoing examples, and thus details are not described herein again.

Referring to FIG. 31, according to yet another aspect of the embodiments of the present disclosure, provided is an electronic device, including a memory 801, a processor 803, a communications interface 805, and a communications bus 807, wherein a computer program capable of running on the processor 803 is stored in the memory 801, and the memory 801 and the processor 803 perform communication with the communication interface 805 via the communication bus 807, and the processor 803, when executing the computer program, implements the above gum line extraction method and/or the manufacturing method of the dental device and/or the tooth arrangement method of the three-dimensional tooth model. Optionally, for specific examples in the present embodiment, reference may be made to the examples described in the above embodiments, and thus details are not described in the present embodiment again.

Those ordinary skill in the art may understand that the structure shown in FIG. 31 is merely schematic, a device for implementing the tooth arrangement method of the three-dimensional tooth model may be a terminal device, and the terminal device may be terminal devices such as a smart phone (e.g., an Android mobile phone, an iOS mobile phone, or the like), a tablet computer, a palmtop computer, a mobile Internet Device (MID), a PAD, or the like. FIG. 31 does not limit the structure of the electronic device, for example, the electronic device may further include more or fewer components (e.g., network interfaces, display apparatuses, or the like) than those shown in FIG. 31, or have different configurations from shown in FIG. 31.

Those ordinary skilled in the art may understand that all or some of the steps in various methods in the above embodiments may be completed by instructing related hardware of the terminal device by means of a program, the program may be stored in a computer-readable storage medium, and the storage medium may include a flash disk, an ROM, an RAM, a magnetic disk, an optical disk, etc.

According to still another aspect of the embodiments of the present disclosure, further provided is a computer-readable storage medium, wherein a computer program is stored in the computer-readable storage medium, and the computer program, when run by a processor, implements the steps in the tooth arrangement method of the three-dimensional tooth model.

Optionally, in the present embodiment, those ordinary skilled in the art may understand that all or some of the steps in various methods in the above embodiments may be completed by instructing related hardware of the terminal device by means of a program, the program may be stored in a computer-readable storage medium, and the storage medium may include a flash disk, a read-only memory (ROM), a random access memory (RAM), a magnetic disk, an optical disk, etc.

The sequence numbers of the above embodiments of the present disclosure are merely for description, and do not represent the advantages and disadvantages of the embodiments.

When an integrated unit in the above embodiments is implemented in the form of a software functional unit and is sold or used as an independent product, it may be stored in the computer-readable storage medium. Based on this understanding, the technical solutions of the present disclosure substantially, or the parts contributing to the prior art, or all or part of the technical solutions may be implemented in the form of a software product, the computer software product is stored in a storage medium, and includes several instructions for enabling one or more computer devices (which may be a personnel computer, a server, or a network device or the like) to execute all or part of the steps of the methods in various embodiments of the present disclosure.

In the above embodiments of the present disclosure, the description of each embodiment has its own emphasis, and for parts which are not described in detail in a certain embodiment, reference may be made to related descriptions in other embodiments.

In the several embodiments provided in the present disclosure, it should be understood that the disclosed client may implemented in other manners. The apparatus embodiments described above are merely exemplary, for example, the division of the units is only a logic function division, there may be other division manners in practical implementations, for example, a plurality of units or components may be combined or integrated into another system, or some features may be omitted or not executed. From another point of view, the displayed or discussed mutual coupling or direct coupling or communication connection may be an indirect coupling or communication connection between units or modules by means of some interfaces, and may be in electrical, mechanical or other forms.

The units described as separate components may be separated physically or not, components displayed as units may be physical units or not, that is, may be located in one place, or may be distributed on a plurality of network units. Part or all of the units may be selected to implement the objectives of the solutions in the present embodiment according to actual requirements.

In addition, the functional units in the embodiments of the present disclosure may be integrated in one processing unit, or the units singly exist physically, or two or more units are integrated in one unit. The integrated unit may be implemented in the form of hardware, and may also be implemented in the form of a software functional unit.

The above descriptions are only preferred implementations of the present disclosure, it should be noted that, for those ordinary skilled in the art, several improvements and modifications may be made without departing from the principles of the present disclosure, and these improvements and modifications should also be considered as the protection scope of the present disclosure.

### INDUSTRIAL APPLICABILITY

The solutions provided in the embodiments of the present disclosure may be applied to the technical field of orthodontics of teeth. In the embodiments of the present disclosure, a three-dimensional tooth model is acquired, wherein the three-dimensional teeth model includes a plurality of polygonal patches; an edge category of an edge of the plurality of polygonal patches is determined, wherein the edge category includes a tooth edge and a gum edge; and a gum line of the three-dimensional tooth model is determined according to the edge categories of the edge. The present disclosure solves the technical problem that the gum line of the tooth model cannot be accurately and efficiently extracted, and achieves the technical effect of accurately and efficiently extracting the gum line of the tooth model. In addition, the present disclosure further uses a method of acquiring a fitting feature point of each tooth in the three-dimensional tooth model; performing fitting according to a position relationship of the tooth relative to the three-dimensional tooth model and the fitting feature point, to obtain a dental arch curve and a torque angle curve; determining a target point closest to each fitting feature point on the dental arch curve, and determining the position where the target point is located as a target position of the corresponding tooth; obtaining a target pose of each tooth corresponding to the target position according to the dental arch curve and the torque angle curve; and moving the tooth to the corresponding target position, and adjusting the pose of the tooth to the corresponding target pose. In the method, during the process of performing tooth arrangement on the teeth in the three-dimensional tooth model, the dental arch curve and the torque angle curve may be determined by the fitting feature point, the target point closest to the fitting feature point is determined on the dental arch curve, the tooth is moved to the target point on the dental arch curve, and the pose of the tooth is adjusted to the target pose, thereby achieving the purpose of automatic tooth arrangement, thus solving the technical problem of low manual tooth arrangement efficiency.

What is claimed is:

1. A gum line extraction method, comprising:
obtaining a three-dimensional tooth model, wherein the three-dimensional tooth model comprises a plurality of polygonal patches;
determining an edge category of at least one edge of the polygonal patch, wherein the edge category comprises a tooth edge and a gum edge;
determining a patch category of the polygonal patch according to the edge category of the at least one edge of the polygonal patch, wherein the patch category comprises a tooth patch and a gum patch;
determining a common edge of adjacent polygonal patches of different patch categories; and
determining a gum line of the three-dimensional tooth model according to a plurality of common edges.

2. The method as claimed in claim 1, wherein determining the edge category of the edge of the plurality of polygonal patches comprises:
acquiring a target feature of a first edge of the plurality of polygonal patches, wherein the target feature comprises a geometric feature; and
determining the edge category of the first edge of the plurality of polygonal patches according to the target feature.

3. The method as claimed in claim 2, wherein the target feature of the edge of the plurality of polygonal patches comprises at least one of the following:
a dihedral angle between a first polygonal patch and a second polygonal patch that share the first edge, a first curvature value of a first vertex of the first edge, a second curvature value of a second vertex of the first edge, a first distance to the first edge from a vertex of the first polygonal patch that is away from the first edge, a second distance to the first edge from a vertex of the second polygonal patch that is away from the first edge, a first angle opposite to the first edge of the first polygonal patch, and a second angle opposite to the first edge of the second polygonal patch.

4. The method as claimed in claim 2, wherein the target feature of the edge of the plurality of polygonal patches comprises at least one of the following:
a dihedral angle between a first polygonal patch and a second polygonal patch that share the first edge, a first curvature value of a first vertex of the first edge, a second curvature value of a second vertex of the first edge, a first spatial coordinate of the first vertex, a second spatial coordinate of the second vertex, the length of the first edge, an included angle between the first edge and an adjacent edge, a first distance to the first edge from a vertex of the first polygonal patch that is away from the first edge, a second distance to the first edge from a vertex of the second polygonal patch that is away from the first edge, a first angle opposite to the first edge of the first polygonal patch, a second angle opposite to the first edge of the second polygonal patch, a first normal of the first vertex, a second normal of the second vertex, a normal of the first polygonal patch, and a normal of the second polygonal patch.

5. The method as claimed in claim 2, wherein determining the edge category of the edge of the plurality of polygonal patches according to the target feature comprises:
performing dimension raising on the target feature with an initial dimension of the edge to obtain a first feature with a first dimension;
performing dimension reduction on the first feature to obtain a second feature with a target dimension; and
determining that the edge is a tooth edge or a gum edge according to a magnitude of a value of the second feature.

6. The method as claimed in claim 1, wherein determining the patch category of the polygonal patch according to the edge category of the at least one edge of the polygonal patch comprises:
when the number of gum edges is greater than the number of tooth edges in the polygonal patch, determining that the polygonal patch is the gum patch; and when the number of gum edges is less than the number of tooth edges in the polygonal patch, determining that the polygonal patch is the tooth patch.

7. The method as claimed in claim 1, wherein the determining a gum line of the three-dimensional tooth model according to a plurality of the common edges comprises:
determining a plurality of first feature points based on vertexes of the plurality of the common edges, wherein the first feature points are located on the three-dimensional tooth model;
determining a plurality of target feature points from the plurality of first feature points; and
determining the gum line according to the plurality of target feature points.

8. The method as claimed in claim 7, wherein the determining a plurality of first feature points based on the vertexes of the plurality of the common edges comprises:
in a case where edge contraction processing has been performed on the three-dimensional tooth model, determining a point, closest to the vertex of the common edge, on the unprocessed three-dimensional tooth model as a first feature point.

9. The method as claimed in claim 7, wherein the determining a plurality of first feature points based on vertexes of the plurality of the common edges comprises:
determining the vertexes on the plurality of common edges as the first feature points.

10. The method as claimed in claim 7, wherein the determining a plurality of target feature points from the plurality of first feature points comprises:
determining, as the target feature point, a first feature point whose curvature is greater than a curvature of an adjacent first feature point in the first feature points.

11. The method as claimed in claim 7, wherein determining the gum line according to the plurality of target feature point comprises:
performing an interpolation operation on the plurality of target feature points to obtain interpolated target feature points; and
connecting the interpolated target feature points in sequence to form the gum line.

12. The method as claimed in claim 1, wherein the method further comprises at least one of following:
displaying the gum line by a display screen; or
generating a cutting path for cutting a shell-shaped film according to the gum line.

13. The method as claimed in claim 1, wherein the method further comprises:
executing at least one of smoothing processing and position adjustment processing on the gum line.

14. A manufacturing method of a dental device, comprising:
obtaining a three-dimensional tooth model, wherein the three-dimensional tooth model comprises a plurality of polygonal patches;
determining an edge category of at least one edge of the polygonal patch, wherein the edge category comprises a tooth edge and a gum edge;
determining a patch category of the polygonal patch according to the edge category of the at least one edge of the polygonal patch, wherein the patch category comprises a tooth patch and a gum patch;
determining a common edge of adjacent polygonal patches of different patch categories;
determining a gum line of the three-dimensional tooth model according to a plurality of the common edges;
generating a cutting path for cutting a shell-shaped film according to the gum line; and
cutting the shell-shaped film according to the gum line to obtain the dental device.

15. The manufacturing method as claimed in claim 14, wherein the dental device is an orthodontic device.

16. The manufacturing method as claimed in claim 14, further comprising:
manufacturing a solid tooth model based on the three-dimensional tooth model; and
covering the solid tooth model by a film, and pressing the film to obtain a shell-shaped film covering the solid tooth model.

17. The manufacturing method as claimed in claim 16, wherein the obtaining a three-dimensional tooth model comprises:
performing tooth cutting on a target three-dimensional tooth model;
performing arrangement processing on teeth obtained after the tooth cutting, to obtain the three-dimensional tooth model.

18. The manufacturing method as claimed in claim 17, wherein the performing tooth cutting on a target three-dimensional tooth model comprises:
obtaining a two-dimensional projection image of the target three-dimensional tooth model, and identifying the two-dimensional projection image to obtain a plurality of tooth regions;
determining, in the target three-dimensional tooth model, original seed points corresponding to the plurality of tooth regions;
expanding the original seed points within a preset range to obtain target seed points of teeth in the target three-dimensional tooth model; and
cutting the target three-dimensional tooth model based on the target seed points of the teeth to obtain cut teeth.

19. The manufacturing method as claimed in claim 16, wherein the performing arrangement processing on the teeth obtained after the tooth cutting comprises:
acquiring a fitting feature point of each tooth in the target three-dimensional tooth model;
performing fitting according to a position relationship of the tooth relative to the three-dimensional tooth model and the fitting feature point, to obtain a dental arch curve and a torque angle curve;
determining a target point closest to each fitting feature point on the dental arch curve, and determining a position where the target point is located as a target position of the corresponding tooth;
obtaining a target pose of each tooth corresponding to the target position according to the dental arch curve and the torque angle curve; and
moving the tooth to the corresponding target position, and adjusting a pose of the tooth to the corresponding target pose.

20. The manufacturing method as claimed in claim 14, further comprising:
extracting peak points of the gum line, and pairing the peak points to obtain a peak point combination;
determining a cutting path between teeth in the tooth regions of the three-dimensional tooth model based on the peak point combination;
performing segmentation processing on the tooth regions according to the cutting path, to obtain cut teeth; and
performing arrangement processing on the cut teeth obtained after the tooth cutting, to obtain a new three-dimensional tooth model, wherein the new three-dimensional tooth model corresponds to the shell-shaped film.
